(19) United States
(12) Patent Application Publication
(10) Pub. No.: US 2026/0272499 A1
(43) Pub. Date: Sep. 17, 2026

(54) METHOD AND DEVICE FOR TREATING CELLULITE

(71) Applicant: Venus Concept Inc., Toronto (CA)

(72) Inventors: Almog Zahavi, Tel Aviv (IL); Yoni Iger, Haifa (IL); Anat Kotler, Herzliya (IL); Genadi Denenburg, Holon (IL)

(73) Assignee: Venus Concept Inc., Toronto, ON (CA)

(21) Appl. No.: 18/872,552

(22) PCT Filed: Jun. 6, 2023
(86) PCT No.: PCT/IB2023/055840

§ 371 Date: Dec. 6, 2024

Related U.S. Application Data
(60) Provisional application No. 63/349,600, filed on Jun. 7, 2022.
(60) Provisional application No. 63/349,601, filed on Jun. 7, 2022.
(60) Provisional application No. 63/349,604, filed on Jun. 7, 2022.
(60) Provisional application No. 63/349,602, filed on Jun. 7, 2022.

(51) Int. Cl. A61B 17/3205; A61B 17/00; A61B 17/02; A61B 18/00; A61B 18/12; A61B 18/14; A61B 34/10; A61B 34/30

(52) U.S. Cl. CPC A61B 17/32053; A61B 17/0218; A61B 18/1206; A61B 18/1402; A61B 34/10; A61B 34/30; A61B 2017/00761; A61B 2017/00893; A61B 2018/00464; A61B 2018/00577; A61B 2018/00589; A61B 2018/00982; A61B 2018/00994; A61B 2034/107

(57) ABSTRACT

A method for treating cellulite on a patient's skin includes identifying at least one region of said patient's skin with cellulite, and forming at least one interference of at least one tissue portion in said region of skin, thereby generating at least one septum in said region of said patient's skin. The step of generating at least one septum in said region of said patient's skin treats cellulite.

### CROSS-REFERENCE TO RELATED APPLICATION

The present application claims priority to U.S. Provisional Application No. 63/349,600 filed Jun. 7, 2022, the entire contents of which are incorporated herein by reference.

### BACKGROUND OF THE INVENTION

Excess tissue and skin laxity are of wide concern in aesthetic medicine. The present invention relates specifically to cellulite (also known as gynoid lipodystrophy, nodular liposclerosis, edematofibrosclerotic panniculopathy, panniculosis, adiposis edematosa, demopanniculosis deformans or status protrusus cutis) and method for treatment thereof.

Moreover, there is a need for proactive treatment modalities that prevent future or reoccurrence of cellulite and which are easy and effective to use.

It has been reported that more than 85% of women have cellulite thus suggesting that cellulite is a physiologic rather than a pathologic condition. Cellulite can be described as the herniation of subcutaneous fat within fibrous connective tissue. This fat loading can lead to stress on connective tissue located between fat lobulas and is expressed as dimpling of the skin. Such dimpling is much more common in women than men due to the orientation of subcutaneous fibrous structures (septa or septae) defining chamber-like structures containing fat cells. In fact, it is this structure that is believed to cause the appearance of cellulite more than being overweight. Often, cellulite appears on the pelvic region including the buttocks, lower limbs and abdomen.

Subdermal or hypodermal fat layers are contained between dermal layers and fascia and are connected by septa which act as structural stabilizing connective tissue between the dermal layer and the fascia. In men, the septa are arranged more randomly and densely and are oriented in a more crisscrossed (X-shaped) configuration while the septa in women are generally more parallel in arrangement and perpendicular to skin surface (see FIG. 20).

Moreover, women with cellulite have exhibited thinning of the overlining dermal zone with thickening of the septa in the regions of cellulite and tensioning of septa highlights cellulite. An increase in fluid retention and/or proliferation of adipose tissue in such subdermal fat layers can further result in the appearance of cellulite where the septa is maintaining a first distance between dermal layers, thus creating dimples, whereas pockets between septa bulge.

Over time, the septa may initially be stretched, then eventually stabilized and harden thus retaining tissue layers at fixed distances between anchoring points of said septa, but pockets between such septa may be expanded thus further pushing upwards dermal and epidermal layers and further adding to the appearance of cellulite.

Various approaches have been taken to treat or address cellulite. Early treatments involved attempts at increasing circulation and lymphatic drainage as well as fat catabolic processes and oxidation in areas exhibiting cellulite. In some, hyaluronic acid and aminophylline were substances provided (e.g., injected) in the target areas to solidify of connective tissue and thus, to reduce cellulite. Other approaches involved applying dermatological creams or other supplements to confront cellulite. These approaches could be supplemented by massage or massage was used alone for the purpose of promoting increased fat reabsorption or lymphatic drainage of fluids and toxins from the treated areas. Ultrasound has also been proposed to disrupt boundary layers of subcutaneous tissues and fat and has been used per-se or in combination with liposuction. Low acoustic pressure in combination with the increase of microbubbles their collapse which results in the destruction of fat cells, has also been employed to reduce the appearance of cellulite, as has been the use of other energies such as lasers and radio frequency to increase metabolism or destruct fat deposits. Such approaches have been characterized by limited or unpredictable results.

More recently, the cutting of septa with blades or needles in the hypodermal region has been employed. Prior approaches have been found to be labor intensive and very traumatic to the tissue-leading to bleeding, bruising, tough tissue nodules, skin tissue folds, long and painful recoveries as well as inconsistent results.

Accordingly, there is still a long felt need for effective and efficient approaches to treating, minimizing or eliminating cellulite with simple systems that provide long lasting results with minimized trauma. These approaches should be associated with predictable results and be relatively easy to employ.

### SUMMARY OF THE INVENTION

In aesthetic medicine, elimination of excess tissue and/or skin laxity is an important concern that affects more than 25% of the U.S. population. Conventional surgical therapies (e.g., a face lift, brow lift, or breast lift) can be effective but are often invasive, inconvenient, and expensive, while scarring limits their applicability.

Removing 5%-15% of skin in an area through excising a multitude of <1 mm diameter cores of dermis and applying directional compression elastic bandages has been shown to provide skin tightening that can be tuned in a desired direction without (noticeable) scarring. An automated robotic dermal micro-coring system with machine vision and robotic precision delivers accuracy, repeatability, and efficiency that provides high value to medical clinics.

Methods using energy sources (e.g., laser, non-coherent light, radiofrequency, or ultrasound) can be effective at improving the architecture and the texture of the skin but are much less effective at tightening the skin or reducing skin laxity. Neurotoxins, such as botulinum toxin, reduce the formation of dynamic wrinkles by paralysis of the injected muscles, but such toxins have minimal or no effect on skin tightness or laxity. Finally, dermal fillers, such as hyaluronic acid, are provided (e.g., injected) in the dermal layer to smooth out wrinkles and improve contours, but such fillers do not tighten or reduce laxity of the skin. Thus, surgical therapies remain the gold standard for lifting and/or tightening skin.

Rotational Fractional Resection (“RFR”) is a procedure which may be used to achieve focal aesthetic contouring by removing fractions of lax skin and excess fat tissue from a patient. Skin may be removed by the use of a rotating micro-coring punch, which is a hollow, sharpened tube which excises full thickness dermal resections. Such punch has been adapted to treat, among other conditions, scars, acne scars, lines, wrinkles, stretch marks, melasma, and to improve skin texture and tighten the skin. As the punch create tiny diameter punctures in the skin; such puncture triggers the body's wound healing process; thereby give the treated area healing process with less discoloration and/or deformation and greater smoothness of the surface.

However, such methods are not problem-free and there is still a need to enhance efficacy thereof. Thus, there is a need for improved methods and devices that increase the effectiveness of such minimally-invasive techniques. Furthermore, there is still a long felt need for an automated including robotic system for dermal micro-coring to be used in minimally invasive directional skin tightening procedures.

This invention relates to methods and devices for skin treatment. More specifically, this invention relates to methods and devices for cellulite treatment.

It is one object of the present invention to provide a method for treating cellulite on a patient's skin so to resolve cellulite phenotype appearance, comprising steps of:
i. identifying at least one region of said patient's skin with expressions of cellulite; and,
ii. forming at least one interference (e.g., coring) of at least a portion of said tissue portion in said region of skin thereby generating at least one septum in said region of said patient's skin;
wherein said step of generating at least one septum in said region of said patient's skin is conducted by said excision of at least one portion of skin tissue and treats expressions of cellulite.

It is another object of the present invention to provide the method as defined above, wherein said step of forming at least one interference of at least one tissue portion comprising at least one step selected from a group consisting of step of excising at least one tissue portion; step of coring at least one tissue portion; step of incision of at least one tissue portion and any combination thereof.

It is another object of the present invention to provide the method as defined above, wherein said step of forming at least one interference of at least one tissue portion at least a portion of said tissue portion is performed up to the fascia tissue of said patient.

It is another object of the present invention to provide the method as defined above, wherein said step of forming at least one interference of at least one tissue portion results in generating a plurality of septa in said region of skin, each at a different angle, relative to each other and said skin.

It is another object of the present invention to provide the method as defined above, wherein said step of forming at least one interference of at least one tissue portion is performed at an angle A with respect to the surface of said region of skin.

It is another object of the present invention to provide the method as defined above, wherein said angle A is in the range of about 0 to about 90 degrees.

It is another object of the present invention to provide the method as defined above, wherein said step of forming at least one interference of at least one tissue portion results in the generation of crisscross structure in said at least one tissue portion.

It is another object of the present invention to provide the method as defined above, wherein said step of identifying at least one region of said patient's skin with expressions of cellulite additionally comprising step of scanning said region of said patient's skin.

It is another object of the present invention to provide the method as defined above, additionally comprising steps of storing said scanned data; and, analyzing thereof so that the efficacy of a treatment can be assessed.

It is another object of the present invention to provide the method as defined above, additionally comprising step of providing recommendations as to where, on said region of skin, to produce said step of forming at least one interference of at least one tissue portion, based on calculated algorithm or accumulated data of safety and efficacy of a treatments of a plurality of patients.

It is another object of the present invention to provide the method as defined above, further comprising step of confirming that the generated septa is associated with the alleviation of said expression of cellulite.

It is another object of the present invention to provide the method as defined above, further comprising step of, if expression of cellulite remains, engaging in additional step of forming at least one interference of at least one tissue portion in said region of skin.

It is another object of the present invention to provide the method as defined above, additionally comprising step of applying contraction or expansion tension to said region of skin tissue before and/or after said step of forming at least one interference of at least one tissue portion.

It is another object of the present invention to provide the method as defined above, wherein said application of contraction or expansion tension to said region of skin tissue is provided by means such as Tegaderm® or pressure bandages.

It is another object of the present invention to provide the method as defined above, wherein said step of applying tension therebetween said two portions applies force in the range of 0 N/mm²-50 N/mm².

It is another object of the present invention to provide the method as defined above, wherein said tension applied in said step of applying tension therebetween said two portions is adjustable based on at least one parameter selected from a group consisting of skin type, age of the patient, type of treatment, anatomy, lesion condition, treated anatomy and any combination thereof.

It is another object of the present invention to provide the method as defined above, wherein said step of applying tension therebetween said two portions is performed at a direction selected from a group consisting of x-, y-, and/or z-direction and any combination thereof.

It is another object of the present invention to provide the method as defined above, wherein said step of forming at least one interference of at least one tissue portion in said region of skin is performed by means selected from a group consisting of mechanical means including rotational means, application of temperature to heat, application of temperature to heat and evacuate tissue, application of laser, pulsed electromagnetic field, application of heat to accelerate collagen synthesis in the tissue, RF, coblation, coagulation, ablation, insertion of threads, microwave energy, ultrasound, application of any other type of energy and any combination thereof.

It is another object of the present invention to provide the method as defined above, wherein said step of forming at least one interference of at least one tissue portion in said region of skin is performed by a system comprising at least one robotic arm, said at least one robotic arm comprising at least one skin interference instrument.

It is another object of the present invention to provide the method as defined above, wherein said at least one skin interference instrument comprising at least one selected from a group consisting of at least one needle, at least one punch, at least one thread and any combination thereof; said at least one skin interference instrument is configured to contact a surface of the skin to destruct (e.g., generate holes) the skin tissue.

It is another object of the present invention to provide the method as defined above, wherein at least a portion of said at least one skin interference instrument is disposable.

It is another object of the present invention to provide the method as defined above, wherein at least two skin interference instruments are adapted to penetrate said skin either in a simultaneously or sequentially manner.

It is another object of the present invention to provide the method as defined above, wherein at least two skin interference instruments are characterized by either a similar or substantially different cross section area.

It is another object of the present invention to provide the method as defined above, wherein said at least one skin interference instrument is adapted to penetrate said skin to a depth of 1 to 20 mm.

It is another object of the present invention to provide the method as defined above, wherein said at least one skin interference instrument is characterized by a diameter of 0.15 mm-2.0 mm.

It is another object of the present invention to provide the method as defined above, wherein said cross section area is selected from a group consisting of circular, rectangular, triangular, hexagonal, oval, staggered rows, parallel rows, a spiral pattern, a square or rectangular pattern, a radial distribution and any combination thereof.

It is another object of the present invention to provide the method as defined above, wherein said system additionally comprising at least one controller adapted to control the positioning and orientation of said at least one robotic arm relatively to said skin area.

It is another object of the present invention to provide the method as defined above, wherein said controller comprising at least one engine adapted to control at least one parameter selected from a group consisting of the rotation, translation, angle of said at least one robotic arm relatively to said skin, exact location of impact, depth of penetration, coverage rate, the diameter of at least one excised tissue multiplied by number of cores (coverage rate), different areas of said skin to be treated and any combination thereof.

It is another object of the present invention to provide the method as defined above, wherein said parameters are adjusted manually by the operator or automatically by said controller.

It is another object of the present invention to provide the method as defined above, wherein said parameters are real time adjusted.

It is another object of the present invention to provide the method as defined above, wherein said rotation is at a speed in the range of 1000-10000 RPM.

It is another object of the present invention to provide the method as defined above, wherein said translation is at a speed in the range of 0-3000 mm/sec.

It is another object of the present invention to provide the method as defined above, wherein said translation of said at least one robotic arm relatively to said skin changes as said at least one robotic arm gets closer to said skin.

It is another object of the present invention to provide the method as defined above, wherein said rotation of said at least one robotic arm changes as said at least one robotic arm gets closer to said skin and penetrates said skin.

It is another object of the present invention to provide the method as defined above, wherein each one skin interference instrument rotates individually in a predefined direction in a predetermined speed.

It is another object of the present invention to provide the method as defined above, wherein at least two of said at least one skin interference instrument rotate simultaneously.

It is another object of the present invention to provide the method as defined above, wherein each one skin interference instrument translates individually.

It is another object of the present invention to provide the method as defined above, wherein at least two of said skin interference instruments translate simultaneously.

It is another object of the present invention to provide the method as defined above, wherein the distance between each pair of neighboring skin interference instruments is configured to vary and be adjustable either before or during treatment.

It is another object of the present invention to provide the method as defined above, wherein said controller comprises a stopper adapted to limit the depth to which at least a portion of said skin interference instrument penetrates said skin.

It is another object of the present invention to provide the method as defined above, additionally comprising step of sensing contact with said skin.

It is another object of the present invention to provide the method as defined above, wherein said angle of said at least one robotic arm is in the range of about 0 to about 90 degrees.

It is another object of the present invention to provide the method as defined above, wherein said controller is adapted to define at least one no-fly zone; said no-fly zone being defined as an area to which said system provides no treatment.

It is another object of the present invention to provide the method as defined above, further comprising at least one force sensor to determine when said at least one skin interference element breaks (i.e., penetrates) the skin.

It is another object of the present invention to provide the method as defined above, additionally comprising step of delivering additives to the skin.

It is another object of the present invention to provide the method as defined above, wherein said additives are selected from a group consisting of threads, therapeutic agents, anesthesia, saline solution growth factors, platelet-derived growth factor (PDGF), transforming growth factor beta (TGF-β), fibroblast growth factor (FGF), epidermal growth factor (EGF), and keratinocyte growth factor); one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins; or one or more vitamins, hyaluronic acid, collagen, low melting agarose (LMA), polylactic acid (PLA), and/or hyaluronic acid, hyaluranon); a photosensitizer (e.g., Rose Bengal, riboflavin-5-phosphate (R-5-P), methylene blue (MB), N-hydroxypyridine-2-(1H)-thione (N-HTP), a porphyrin, or a chlorin, as well as precursors thereof); a photochemical agent, 1,8 naphthalimide); a synthetic glue (e.g., a cyanoacrylate adhesive, a polyethylene glycol adhesive, or a gelatin-resorcinol-formaldehyde adhesive), a biologic sealant and any combination thereof.

It is another object of the present invention to provide the method as defined above, wherein said system additionally comprising at least one imaging subsystem adapted to guide said at least one skin interference instrument.

It is another object of the present invention to provide the method as defined above, wherein said imaging subsystem comprises at least one selected from a group consisting at least one camera, under-skin imaging such as ultrasound-based imaging, OCT and any combination thereof.

It is another object of the present invention to provide the method as defined above, wherein said system additionally comprising at least one subsystem selected from a group consisting of (a) vacuum subsystem adapted to apply suction to remove excising portions of said skin tissue; (b) at least one retainer, in communication with at least one excisor configured to produce a plurality of excised tissue portions, adapted to contain said excised tissue, to avoid the use of vacuum; (c) any combination thereof.

It is another object of the present invention to provide the method as defined above, wherein said skin is part of a treatment area selected from a group consisting of buttocks, lower limbs, abdomen and any combination thereof.

It is another object of the present invention to provide the method as defined above, wherein said system utilizes at least one selected from a group consisting of mechanical visualization, OCT, Ultrasound, machine learning algorithms, artificial intelligence, image processing and any combination thereof to efficiency select the preferred location of the tissue to be treated to enhance outcome of said treatment.

It is another object of the present invention to provide the method as defined above, additionally comprising step of stabilizing said at least one septum (one or more septa) generated.

It is another object of the present invention to provide the method as defined above, wherein said step of stabilizing is performed by application of at least one selected from a group consisting of RF energy, pulsed electromagnetic field, microwave, optical energy, ultrasound, and any combination thereof.

It is another object of the present invention to provide the method as defined above, additionally comprising step of application of at least one thread after and/or before and/or during said step of forming at least one interference of at least one tissue portion in said region of skin; thereby stabilizing said at least one septum generated.

It is another object of the present invention to provide the method as defined above, additionally comprising step of providing at least one cutting element adapted to grind said excised tissue so as to facilitate extraction thereof.

It is another object of the present invention to provide the method as defined above, additionally comprising step of communicating said at least one skin interference instrument with at least one RF generator, adapted to apply RF energy to the skin and tissue, so as to fractional ablate/coagulate the tissue.

It is another object of the present invention to provide the method as defined above, wherein said application of RF energy is either simultaneously or sequentially with the formation of at least one interference.

It is another object of the present invention to provide the method as defined above, additionally comprising step of providing at least one dynamic magnetic field pulses to said skin.

It is another object of the present invention to provide the method as defined above, wherein said electromagnetic pulses and said RF energy are provided simultaneously to said skin.

It is another object of the present invention to provide the method as defined above, wherein said RF energy results in heating said skin.

It is another object of the present invention to provide the method as defined above, additionally comprising step of application of at least one energy selected from a group consisting of laser, pulsed electromagnetic field, RF, coblation, coagulation, ablation, microwave energy, ultrasound, application of any other type of energy and any combination thereof; is either simultaneously or sequentially applied with the formation of at least one interference.

It is another object of the present invention to provide the method as defined above, wherein at least one of the following is being held true (a) the shape of said electromagnetic pulse is selected from the group consisting of square wave, a sine wave, a triangular wave, sawtooth wave, ramp waves, spiked wave or any combination thereof; (b) the magnetic field intensity B of each pulse applied by said pulsed electromagnetic frequency generator ranges between about 0 and about 3 Tesla; (c) the magnetic field intensity B of each pulse applied by said pulsed electromagnetic frequency generator ranges between about 0 to 40 Gauss; (d) the duration of each pulse applied by said pulsed electromagnetic frequency generator ranges between about 3 and about 1000 milliseconds; (e) the frequency F applied by the pulses of said pulsed electromagnetic frequency generator ranges between about 1 Hz and about 40 MHz; (f) the energy E applied by the pulses of said pulsed electromagnetic frequency generator ranges between about 1 and about 150 watts per pulse or any combination thereof; (g) the frequency F applied by the pulses applied by said step of applying pulsed electromagnetic therapy to said region to be higher than about 1 and lower than about 1M Hz; (h) the frequency F applied by said electromagnetic field pulses ranges between 1 Hz and 50 Hz; (i) the frequency of said RF energy ranges between 200 kHz and 10 MHz; (j) the power P applied by said RF energy pulses ranges between 1 W and 100 W of RMS average power; and any combination thereof.

It is another object of the present invention to provide the method as defined above, additionally comprising step of cooling said skin.

It is another object of the present invention to provide the method as defined above, additionally comprising step of providing at least one treatment substance to the treatment area.

It is another object of the present invention to provide the method as defined above, additionally comprising step of at least partially severing at least one septum.

It is another object of the present invention to provide the method as defined above, wherein said step of at least partially severing at least one septum is performed by said step of forming at least one interference of at least one tissue portion in said region of skin.

It is another object of the present invention to provide a system of treating expressions of cellulite on a patient's skin, comprising:
(i) means for identifying at least one region of said patient's skin with expressions of cellulite; and,
(ii) means for forming at least one interference of at least one tissue portion in said region of skin thereby generating at least one septa in said region of said patient's skin;
wherein said at least one septa generated in said region of said patient's skin treats expressions of cellulite.

It is another object of the present invention to provide the system as defined above, wherein said means of forming at least one interference of at least one tissue portion comprising means of excising at least one tissue portion; means of coring at least one tissue portion; means of incision of at least one tissue portion and any combination thereof.

It is another object of the present invention to provide the system as defined above, wherein said step of forming at least one interference of at least one tissue portion is performed up to the depth of the fascia tissue of treated area of said patient.

It is another object of the present invention to provide the system as defined above, wherein said step of forming at least one interference of at least one tissue portion results in generating a plurality of septa in said region of skin, each at a different angle, relative to each other and said skin.

It is another object of the present invention to provide the system as defined above, wherein said means of forming at least one interference of at least one tissue portion is performed at an angle A with respect to the surface of said region of skin.

It is another object of the present invention to provide the system as defined above, wherein said angle A is in the range of about 0 to about 90 degrees.

It is another object of the present invention to provide the system as defined above, wherein said means of forming at least one interference of at least one tissue portion results in the generation of crisscross structure in said at least one tissue portion.

It is another object of the present invention to provide the system as defined above, wherein said step of identifying at least one region of said patient's skin with expressions of cellulite additionally comprising step of scanning said region of said patient's skin.

It is another object of the present invention to provide the system as defined above, additionally comprising step of storing said scanned data; and, analyzing thereof so that the efficacy of a treatment can be assessed.

It is another object of the present invention to provide the system as defined above, additionally comprising step of providing recommendations as to where on said region of skin, based on efficacy of a treatments of a plurality of patients.

It is another object of the present invention to provide the system as defined above, further comprising step of confirming that the generated septae is associated with the alleviation of expression of cellulite.

It is another object of the present invention to provide the system as defined above, additionally comprising step of applying stabilization, contraction or expansion tension to said region of skin tissue before and/or after said step of forming at least one interference of at least one tissue portion.

It is another object of the present invention to provide the system as defined above, wherein said application of contraction or expansion tension to said region of skin tissue is provided by at least one selected from a group consisting of Tegaderm®, pressure bandages and any combination thereof.

It is another object of the present invention to provide the system as defined above, wherein said directional skin tightening is performed at a direction selected from a group consisting of the x-, y-, and/or z-direction and any combination thereof.

It is another object of the present invention to provide the system as defined above, wherein said means of forming at least one interference of at least one tissue portion in said region of skin comprising means selected from a group consisting of mechanical means, application of temperature to heat and evacuate tissue, application of temperature to heat, application of laser, insertion of threads, application of heat to accelerate collagen synthesis in the tissue, RF, pulsed electromagnetic field, coblation, ablation, coagulation, microwave energy, ultrasound, application of any other type of energy and any combination thereof.

It is another object of the present invention to provide the system as defined above, wherein said means of forming at least one interference of at least one tissue portion in said region of skin comprising a system comprising at least one robotic arm, said at least one robotic arm comprising at least one skin interference instrument.

It is another object of the present invention to provide the system as defined above, wherein said at least one skin interference instrument comprising a plurality of punches configured to contact a surface of the skin to generate holes in the skin tissue.

It is another object of the present invention to provide the system as defined above, wherein said at least one skin interference instrument are disposable.

It is another object of the present invention to provide the system as defined above, wherein said plurality of said at least one skin interference instrument are adapted to penetrate said skin either in a simultaneously or sequentially manner.

It is another object of the present invention to provide the system as defined above, wherein said plurality of said at least one skin interference instrument are characterized by either a similar or substantially different cross section area.

It is another object of the present invention to provide the system as defined above, wherein said plurality of said at least one skin interference instrument are adapted to penetrate said skin to a depth of 1 to 20 mm.

It is another object of the present invention to provide the system as defined above, wherein at least a portion of said plurality of said at least one skin interference instrument are characterized by a diameter of 0.15 mm-2.0 mm.

It is another object of the present invention to provide the system as defined above, wherein said cross section area is selected from a group consisting of circular, rectangular, triangular, hexagonal, oval, staggered rows, parallel rows, a spiral pattern, a square or rectangular pattern, a radial distribution and any combination thereof.

It is another object of the present invention to provide the system as defined above, wherein said system additionally comprising at least one controller adapted to control the positioning and orientation of said at least one robotic arm relatively to said skin area.

It is another object of the present invention to provide the system as defined above, wherein said controller comprising at least one engine adapted to control at least one parameter selected from a group consisting of the rotation, translation, angle of said at least one robotic arm relatively to said skin, exact location of impact, depth of penetration, coverage rate, the diameter of at least one excised tissue multiplied by number of cores, different area of said skin to be treated and any combination thereof.

It is another object of the present invention to provide the system as defined above, wherein said parameters are adjusted manually by the operator or automatically by said controller.

It is another object of the present invention to provide the system as defined above, wherein said parameters are real time adjusted.

It is another object of the present invention to provide the system as defined above, wherein said rotation is at a speed in the range of 1000-10000 RPM.

It is another object of the present invention to provide the system as defined above, wherein said translation is at a speed in the range of 0-3000 mm/sec.

It is another object of the present invention to provide the system as defined above, wherein said translation of said at least one robotic arm relatively to said skin changes as said at least one robotic arm gets closer to said skin.

It is another object of the present invention to provide the system as defined above, wherein said rotation of said at least one robotic arm changes as said at least one robotic arm gets closer to said skin and penetrates said skin.

It is another object of the present invention to provide the system as defined above, wherein each punch of said plurality of said at least one skin interference instrument rotates individually in a predefined direction in a predetermined speed.

It is another object of the present invention to provide the system as defined above, wherein said plurality of said at least one skin interference instrument rotate simultaneously.

It is another object of the present invention to provide the system as defined above, wherein each punch of said plurality of said at least one skin interference instrument translates individually.

It is another object of the present invention to provide the system as defined above, wherein said plurality of said at least one skin interference instrument translate simultaneously.

It is another object of the present invention to provide the system as defined above, wherein the distance between each of said at least one skin interference instrument can vary and be adjustable either before or during treatment.

It is another object of the present invention to provide the system as defined above, wherein said controller comprising stopping mechanism adapted to limit the depth to which at least a portion of said plurality of said at least one skin interference instrument penetrate said skin.

It is another object of the present invention to provide the system as defined above, additionally comprising at least one sensor adapted to indicate contact with said skin.

It is another object of the present invention to provide the system as defined above, wherein said angle of said at least one robotic arm to said skin is in the range of about 0 to about 90 degrees.

It is another object of the present invention to provide the system as defined above, wherein said controller is adapted to define at least one no-fly zone; said no-fly zone is defined as an area to which said system provides no treatment.

It is another object of the present invention to provide the system as defined above, further comprising at least one force sensor to determine when said at least one skin interference element penetrates the skin.

It is another object of the present invention to provide the system as defined above, wherein said system additionally provide the skin with additives.

It is another object of the present invention to provide the system as defined above, wherein said additives are selected from a group consisting of threads, therapeutic agents, anesthesia, saline solution growth factors, platelet-derived growth factor (PDGF), transforming growth factor beta (TGF-β), fibroblast growth factor (FGF), epidermal growth factor (EGF), and keratinocyte growth factor); one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials ; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins; or one or more vitamins, hyaluronic acid, collagen, low melting agarose (LMA), polylactic acid (PLA), and/or hyaluronic acid, hyaluranon); a photosensitizer (e.g., Rose Bengal, riboflavin-5-phosphate (R-5-P), methylene blue (MB), N-hydroxypyridine-2-(1H)-thione (N-HTP), a porphyrin, or a chlorin, as well as precursors thereof); a photochemical agent, 1,8 naphthalimide); a synthetic glue (e.g., a cyanoacrylate adhesive, a polyethylene glycol adhesive, or a gelatin-resorcinol-formaldehyde adhesive), a biologic sealant and any combination thereof.

It is another object of the present invention to provide the system as defined above, wherein said system additionally comprising at least one imaging subsystem adapted to guide said at least one skin interference instrument.

It is another object of the present invention to provide the system as defined above, wherein said imaging subsystem comprises at least one selected from a group consisting at least one camera, under skin imaging such as ultrasound-based imaging, OCT and any combination thereof.

It is another object of the present invention to provide the system as defined above, wherein said system additionally comprising at least one subsystem selected from a group consisting of (a) vacuum subsystem adapted to apply suction to remove excising portions of said skin tissue; (b) at least one retention element, in communication with at least one of said means for producing a plurality of excised tissue portions, adapted to contain said excised tissue, to avoid the use of vacuum; (c) any combination thereof.

It is another object of the present invention to provide the system as defined above, wherein said skin could be part of a treatment area selected from a group consisting of buttocks, lower limbs, abdomen and any combination thereof.

It is another object of the present invention to provide the system as defined above, wherein said system utilizes at least one selected from a group consisting of mechanical visualization, OCT, Ultrasound, machine learning algorithms, artificial intelligence, image processing and any combination thereof to efficiency select the preferred location of the tissue to be treated to enhance outcome of said treatment.

It is another object of the present invention to provide the system as defined above, additionally stabilizing means, adapted to stabilize said at least one septa generated.

It is another object of the present invention to provide the system as defined above, wherein said stabilizing means are adapted to perform application of at least one selected from a group consisting of RF energy, pulsed electromagnetic field, microwave, optical energy, ultrasound, and any combination thereof.

It is another object of the present invention to provide the system as defined above, additionally comprising means for application of at least one thread to stabilize said at least one septa generated.

It is another object of the present invention to provide the system as defined above, additionally comprising at least one cutting element, integrated within said skin interference instrument, adapted to grind said excised tissue so as to facilitate extraction thereof.

It is another object of the present invention to provide the system as defined above, wherein said at least one skin interference instrument is in communication with at least one RF generator, adapted to apply RF energy to the skin and tissue, so as to fractional ablate/coagulate the tissue.

It is another object of the present invention to provide the system as defined above, wherein said application of RF energy is either simultaneously or sequentially with the formation of interference.

It is another object of the present invention to provide the system as defined above, wherein said at least one skin interference instrument is in communication with at least one pulsed electromagnetic frequency generator.

It is another object of the present invention to provide the system as defined above, wherein said pulsed electromagnetic frequency generator is adapted to provide at least one dynamic magnetic field pulses to said skin.

It is another object of the present invention to provide the system as defined above, wherein said dynamic magnetic field pulses are provided by means of at least one coil.

It is another object of the present invention to provide the system as defined above, wherein said at least one skin interference instrument is at least partially coiled by at least one coil.

It is another object of the present invention to provide the system as defined above, wherein said at least one skin interference instrument is adapted to simultaneously provide both said electromagnetic pulses and said RF energy to said skin.

It is another object of the present invention to provide the system as defined above, wherein said RF energy results in heating said skin.

It is another object of the present invention to provide the system as defined above, wherein application of at least one energy selected from a group consisting of laser, pulsed electromagnetic field, RF, coblation, coagulation, ablation, microwave energy, ultrasound, application of any other type of energy and any combination thereof is either simultaneously or sequentially applied with the formation of interference of said skin.

It is another object of the present invention to provide the system as defined above, wherein at least one of the following is being held true (a) the shape of said electromagnetic pulse is selected from the group consisting of square wave, a sine wave, a triangular wave, sawtooth wave, ramp waves, spiked wave or any combination thereof; (b) the magnetic field intensity B of each pulse applied by said pulsed electromagnetic frequency generator ranges between about 0 and about 3 Tesla; (c) the magnetic field intensity B of each pulse applied by said pulsed electromagnetic frequency generator ranges between about 0 to 40 Gauss; (d) the duration of each pulse applied by said pulsed electromagnetic frequency generator ranges between about 3 and about 1000 milliseconds; (e) the frequency F applied by the pulses of said pulsed electromagnetic frequency generator ranges between about 1 Hz and about 40 MHz; (f) the energy E applied by the pulses of said pulsed electromagnetic frequency generator ranges between about 1 and about 150 watts per pulse or any combination thereof; (g) the frequency F applied by the pulses applied by said step of applying pulsed electromagnetic therapy to said region to be higher than about 1 and lower than about 1M Hz; (h) the frequency F applied by said electromagnetic field pulses ranges between 1 Hz and 50 Hz; (i) the frequency of said RF energy ranges between 200 kHz and 10 MHz; (j) the power P applied by said RF energy pulses ranges between 1 W and 100 W of RMS average power; and any combination thereof.

It is another object of the present invention to provide the system as defined above, additionally comprising at least one temperature sensor.

It is another object of the present invention to provide the system as defined above, additionally comprising a mechanism for skin cooling, adapted to regulate the temperature of the skin.

It is another object of the present invention to provide the system as defined above, wherein the distal end of said at least one skin interference instrument additionally comprising at least one selected from a group consisting of at least one impedance, at least one temperature sensor and any combination thereof.

It is another object of the present invention to provide the system as defined above, wherein said at least one selected from a group consisting of at least one impedance, at least one temperature sensor and any combination thereof is adapted to provide indication as to the depth of penetration of each of said at least one skin interference instrument.

It is another object of the present invention to provide the system as defined above, wherein said at least one skin interference instrument additionally comprising at least one needle, adapted to provide at least one treatment substance to the treatment area.

It is still an object of the present invention to provide the system as defined above, additionally means adapted to at least partially severing at least one septum.

It is lastly an object of the present invention to provide the system as defined above, wherein said means for forming at least one interference of at least one tissue portion in said region of skin, are adapted to at least partially severing at least one septum.

### BRIEF DESCRIPTION OF THE DRAWINGS

FIG. 1 illustrates the general operation of the device of the present invention.

FIG. 2 illustrates a dermal micro-coring process using multiple hollow rotating sharp punch. FIG. 2 illustrates a single punch.

FIGS. 3A-3E illustrate two possible punch rotation drive types: belt driven and friction driven.

FIG. 4 illustrates the dissected skin cores from each punch are pulled up by vacuum.

FIGS. 5A-5B illustrate one arm, each of which utilizes 1 or more punches, as embodied in the system.

FIG. 6 illustrates a top view of the punches. The figures are drafted as coaxial punches.

FIGS. 7-9 illustrate one instrument design configured to spread out punches allowing overlapping patterns.

FIGS. 10A-10B illustrate one embodiment of the stretching/compression device.

FIGS. 11-12 illustrate the short side, according to this embodiment, of the stretching/compression device.

FIGS. 13-14 illustrate the long side, according to this embodiment, of the stretching/compression device.

FIGS. 15-16 illustrate another embodiment of the directional tightening method and device according to the present invention.

FIGS. 17A-17B illustrates histological analysis-cross tissue sections after 0, 2 and 5 weeks post the fractional coring (tissue removal) treatment.

FIGS. 18A and 18B illustrate side and longitudinal views, respectively, of a biological unit removal tool having a movable retention member (retainer or retainer element) in the form of inner tines in a retracted or undeployed state.

FIGS. 19A and 19B illustrate side and longitudinal views of the biological unit removal tool of FIGS. 18A and 18B in a retentive state.

FIG. 20 depicts cross-sections of skin.

### DETAILED DESCRIPTION OF THE INVENTION

This invention relates to methods and devices for the treatment of cellulite. Such treatment is provided by the generation of septa by means of tissue interference (e.g., tissue incision and/or tissue excision).

The present invention relates specifically to cellulite (also known as gynoid lipodystrophy, nodular liposclerosis, edematofibrosclerotic panniculopathy, panniculosis, adiposis edematosa, demopanniculosis deformans or status protrusus cutis) and method for treatment thereof.

It is known that more than 85% of women have cellulite thus suggesting that cellulite is a physiologic rather than pathologic condition. Cellulite can be described as the herniation of subcutaneous fat within fibrous connective tissue that is expressed as dimpling of the skin. This fat loading can lead to stress on connective tissue located between fat lobulas. Such dimpling is more common in women than men due to the orientation of subcutaneous fibrous structures defining chambers containing fat cells. In fact, it is this structure that is believed to cause the appearance of cellulite more than being overweight. Often, cellulite appears on the pelvic region including the buttocks, lower limbs and abdomen.

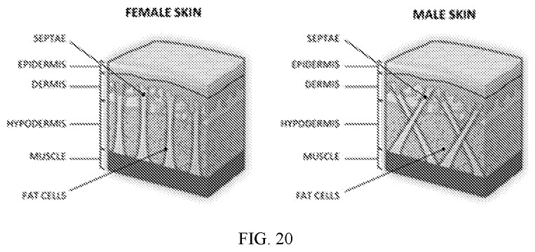

Subdermal fat layers below the epidermis are contained between dermal layers connected by septa which act as connective tissue between the dermal layers. In men, the septa are arranged more randomly and densely oriented in a more polygonal shape (crisscrossed or X-shaped) configuration while the septa in women are generally more parallel in arrangement (see FIG. 20). Such change in the septa's structure result in men having practically no cellulite while women suffer therefrom.

While various approaches have been taken to treat or address cellulite, none gave any real alleviation to the condition. Early treatments involved attempts at increasing circulation and fat oxidation in areas exhibiting cellulite. In some, hyaluronic acid and aminophylline were substances provided (e.g., injected) in the target areas to reduce cellulite. Other approaches involved electroporating the target areas followed by the application of mesotherapy, or applying dermatological creams or other supplements to cellulite. These approaches could be supplemented by massage or massage was used alone for the purpose of promoting increased fat reabsorption or drainage of fluids and toxins in the treated areas. Ultrasound has also been proposed to disrupt subcutaneous tissues and fat and has been used in combination with liposuction. Low acoustic pressure in combination with the infiltration of microbubbles has also been employed to reduce the appearance of cellulite, as has the use of other energies such as lasers and radio frequency. Such approaches have been characterized by limited or unpredictable results.

More recently, the cutting of septa with blades or needles in the subdermal region has been employed. Prior approaches have been found to be labor intensive and very traumatic to the tissue leading to bleeding, bruising, tough tissue nodules, long, painful recoveries and inconsistent results.

The present invention provides an effective and efficient approaches to treating, minimizing or eliminating cellulite with simple systems that minimize trauma. The main concept of the present invention is to tissue engineer the skin region to mimic the male septa structure (namely, the crisscrossed (X-shaped) configuration). Thus, in other words, the present invention generates in the required treatment area a plurality of septa structured as crisscrossed to support the subcutaneous fat and to alleviate in the elimination of cellulite.

According to a preferred embodiment of the present invention the device and method of the present invention discloses the deliberately generation of scar tissue in predetermined locations and at a preterminal density. As scar tissue generated by the present invention results in having substantially the same properties as fibrous septae, such generation of scar tissue is in-fact generation of septa.

More specifically, the present invention relates to tissue engineering and the generation of at least one septa. Such generation of at least one septum is provided by forming at least one interference of at least one tissue portion. Such interference is provided by tissue excision/incision and/or coring and/or any destruction or disruption to the tissue.

Tissue excision/incision and/or coring and/or any destruction or disruption to the tissue can be performed by fractional ablation of the epidermal and/or dermal layer of the skin with at least one hollow coring needle (or punch), by fractional laser ablation, by fractional radiofrequency (also refers to as RF), either by one or multiple RF electrodes, ablation, and/or by fractional ultrasonic ablation (using ultrasound), application of temperature to heat, application of laser, insertion of threads, RF, pulsed electromagnetic field, coblation, ablation, coagulation, application of heat to accelerate collagen synthesis in the tissue, microwave energy, any destruction or disruption to the tissue, application of any other type of energy and any other type of energy, any additives to the tissue and any combination thereof.

Thus, according to one embodiment, the device of the present invention creates interference of the tissue in a predetermined skin portions.

According to one embodiment, the device of the present invention excises patterns of dermal skin cores at desired density, and direction. Optionally, those holes are stabilized by, e.g., application of RF energy, PEMF (pulse electromagnetic pulses), ultrasound energy, microwave etc.

It is noted that stabilization refers hereinafter to the increase of the yield of collagen synthesis, yielding thicker/denser scar tissue.

As another alternative, the remaining holes in the skin are closed using manual compression methods such as compression tape, glue or tunable dressings.

According to one embodiment of the present invention, the device of the present invention is designed for the creation of skin micro-interference in a fractional manner.

According to one embodiment of the present invention, the device of the present invention is designed for the removal of skin micro-cores in a fractional manner.

According to one embodiment of the present invention, the device and method of the present invention can be utilized for the prevention of cellulite. Namely, providing, in advance, the treatment to prevent the appearance of cellulite.

### Definitions

The term “septae” refers hereinafter to bundles of dense connective tissue interconnecting the dermis, through the hypodermis, with the fascia layer. Fibrous septa are like narrow, semi-rigid bands that pull the skin downwards at anchoring points, thus creating the typical effect of cellulite on the surface of the skin, with the accumulation of fast and liquids in between the said septa.

Fibrous septa are among the causes of the “visibility” of cellulite. The very existence and orientation of these structures, together with their thickenings and thinning of dermal zone, under pressure of accumulated fat and hampered lymphatic drainage are part of the structure and function environment of cellulite. Precisely the structure of cellulite is the last frontier for treatments which, thanks to research and innovation, give more long-lasting results without resorting to scalpels. Cellulite affects the overwhelming majority of women, usually after puberty. According to some experts, severing the fibrous septa allows the skin's surface to smooth out; the result is a long-lasting improvement in the appearance of cellulite. As will be discussed hereinafter the present invention discloses means and method to generate new septa to treat cellulite.

Cellulite is thought to be the result of complex mechanisms that are manifested in different ways depending on their evolution in adipose tissue:
increase in adipocytes, increase in the volume of adipose cells, alteration of the shape, cell thickening, neoformation of collagen fibres, encapsulation of degenerated adipocytes, formation of micronodules, evolution into palpable, visible macronodules, formed pressure that hamper lymphatic drainage from hypodermis, hardening of the connective fibrous septae, stretching of the skin upwards and the formation of craters which cause the typical “dimpled” effect.

In women, the fibrous septa are essentially perpendicular to the skin surface in rectangular sections. Therefore, it lacks the ‘mechanical support’ to prevent the formation of cellulite with the accumulation of hypodermal pressure due to its increased volume. On the other hand, in the subcutaneous tissue of men, fibrous septa are arranged in a rhomboid manner with a polygonal shape (crisscross, X-shape), thereby providing support to prevent the creation of septa.

The main role of the connective tissue of the dermis is to provide mechanical strength and elasticity so to maintain structure, isolation and stability of the subcutaneous and deeper zones. The connective tissue is mainly composed of:
collagen which gives tensile strength
elastic tissue that gives the skin the ability to stretch and return to normal

There are 3 structural problems that cause cellulite:
The fibrous septa existence and orientation, including it thickening and stiffening
Fat increases in volume and is trapped and retained in lobules between septae, further pressing and hampering lymphatic drainage
The dermis thins and eases the existence and worsens the appearance of cellulite

The term “scar tissue” refers hereinafter to fibrous tissue that is being formed when normal tissue is affected beyond threshold or destroyed by endogenic or exogenic impacts or interventions such as disease, injury, or surgery. For example, scar tissue is formed as part of the common wound healing process when a wound is formed by a cut, sore, burn, or other skin condition, or when an incision (cut) is made into the skin during surgery. It may also be formed inside the body when certain conditions, such as cirrhosis, cause normal tissue to become fibrotic tissue.

The term “about” refers hereinafter to +/−25% of any recited value.

The term “overlap” refers hereinafter to vertex, facet, cross sectional area and any combination thereof.

The term “Optical coherence tomography (OCT)” refers hereinafter to a non-invasive imaging. In other words, OCT is an imaging technique that uses low-coherence light to capture micrometer-resolution, two-and three-dimensional images from within optical scattering media (e.g., biological tissue). It is used for medical imaging and industrial nondestructive testing (NDT). Optical coherence tomography is based on low-coherence interferometry, typically employing near-infrared light. The use of relatively long wavelength light allows it to penetrate into the scattering medium. Confocal microscopy, another optical technique, typically penetrates less deeply into the sample but with higher resolution.

The term “mechanical visualization” refers hereinafter to either the use of ultrasound or OCT to image the under surface of the treated area skin/tissue. Such mechanical visualization is used to efficiency select the preferred location of the tissue to be treated to enhance outcome of said treatment. It should be noted that according to the present invention the term ‘mechanical visualization’ also includes 2D and 3D cameras for imaging the surface of the treated area skin/tissue.

The term “skin” refers hereinafter to the largest organ of the body. The skin protects us from microbes and the elements, helps regulate body temperature, and permits the sensations of touch, heat, and cold. The skin has several layers:
The epidermis, the outermost layer of skin.
The dermis, beneath the epidermis, contains tough connective tissue, hair follicles, and sweat glands.
The deeper subcutaneous tissue (hypodermis) is made of fat and connective tissue and is up to the fascia.

According to the present invention the term “skin” refers to all 3 layers (up to the fascia) or any portion thereof.

The term “interference” refers hereinafter to either incision/incised tissue and/or excision/excised tissue and/or coring (by means selected from of mechanical means, blades, one or more solid needles, application of temperature to heat and evacuate tissue, application of temperature to heat, application of laser, insertion of threads, pulsed electromagnetic field, RF, coblation, coagulation, ablation, microwave energy, application of heat to accelerate collagen synthesis in the tissue, ultrasound, fractional laser ablation, fractional radiofrequency ablation, coblation, coagulation, microwave energy and/or fractional ultrasonic ablation, application of any other type of energy and any combination thereof).

The term “incised” tissue portion or “incision” refers hereinafter to any destruction or disruption to the tissue such as a cut, abrasion, ablation or coagulation of tissue, including a tissue portion in a skin region, or the act of cutting, abrading, or ablating tissue, in a skin region, or one or more tissue portions. For example, an incision includes any cut, abrasion, or ablation into tissue, which can result in destruction of tissue or a portion thereof and, thereby, produce one or more holes or slits in the skin region. Exemplary methods of forming incised tissue portions or incisions include use of one or more blades, one or more solid needles, fractional laser ablation, fractional radiofrequency ablation, coblation, coagulation, microwave energy and/or fractional ultrasonic ablation, any useful tool for forming tissue destruction or incisions, or any methods and apparatuses described herein.

The term “excised” tissue portion or “excision” refers hereinafter to a removed tissue, including a tissue portion from a skin region, or the act of removing tissue or one or more tissue portions from a skin region. Excision is usually referred to as “to surgically remove”. This term is often used in reference to removing a mass, excision means that tissue is removed, using a scalpel, laser, coblation, coagulation, ablation, ultrasound, microwave energy, RF, application of heat (to evaporate skin portions), mechanical applicator that ‘drills’ (cores) through the skin whilst suction is applies (during the drilling/coring or thereafter) to remove the to be excised skin portion, or any other instrument. For example, an excision includes any removed tissue or tissue portion from a skin region, which can result in excised tissue portions having a particular geometry (e.g., a cylindrical geometry, rectangular, triangle etc. or any arbitrary shape) and produce one or more holes (i.e., negative space created by the removal of tissue) in the skin region. Exemplary methods of forming excised tissue portions or excisions include use of one or more hollow needles (optionally include one or more notches, extensions, protrusions, and/or barbs), one or more microaugers, one or more microabraders, vacuum, any ablative means (including ablative lasers etc.)-may be used for incision and for excision, any useful tool for forming excisions, or any methods and apparatuses described herein.

The term “application of compression forces” refers hereinafter to a physical change in the compression tape (as disclosed hereafter). In this case, the forces applied are compression forces to compress a tape (e.g., TegaDerm®).

The term “application of expansion forces” refers hereinafter to a physical change in the compression tape (as disclosed hereafter). In this case, the forces applied are stretching forces to expand the tape.

It is emphasized that the following disclosure provides an example of interference instrument being coring means (e.g., punches/needles), however, any other example that results in either incision/incised tissue and/or excision/excised tissue and/or coring (by means selected from of mechanical means, blades, one or more solid needles, application of temperature to heat and evacuate tissue, application of temperature to heat, application of laser, insertion of threads, application of heat to accelerate collagen synthesis in the tissue, pulsed electromagnetic field, RF, coblation, coagulation, ablation, microwave energy, ultrasound, fractional laser ablation, fractional radiofrequency ablation, coblation, coagulation, microwave energy and/or fractional ultrasonic ablation, application of any other type of energy and any combination thereof) is within the scope of the present invention.

According to one embodiment of the present invention, the interference mechanism is a single-use disposable cartridge consisting of at least one, up to 0.75 mm in diameter, hollow needles (or punches) inserted into the skin while rotating at about 10000 RPM with a maximum penetration depth of up to 10 mm.

According to one embodiment of the present invention, the interference mechanism is the applications of additives (e.g., threads) inserted into the treated tissue. Thus, according to this embodiment the interference to the skin is performed by insertion (and securing) threads into the skin (up to the fascia tissue) in a predetermined pattern (e.g., amount, density, orientation etc.) so as to mimic the crisscross structure of the male septa.

As described above, this invention further relates to methods and devices for skin treatment. More, specifically, this invention relates to methods and devices for skin interference (e.g., coring, incision/incised tissue and/or excision/excised tissue) that would promote collagen growth in the generation of septa.

Although the device is primarily used for cellulite, it could be utilized in a wide variety of fields e.g., skin laxity, skin resurfacing, cheek wrinkles treatments, wrinkles treatments, folds treatments, acne scars removal, dyschromia treatment, striae treatment, surgical or burn scars removal, cellulite treatment, tattoos removal and any combination thereof.

In particular embodiments, the present invention provides one or more of the following advantages. First, the methods and devices herein enable visualization of results in real time during the course of the treatment. One can envision asking the patient for feedback in real time during the treatment and adjusting the tightening to the patient preference. Second, the methods and devices herein require less skill than that of a surgeon. One can envision treatment of patients in an outpatient setting, rather than requiring an inpatient, surgical setting. Third, the methods and devices herein constitute minimally invasive techniques, which can provide more predictable results and/or lower risk factors than that for more invasive techniques (e.g., plastic surgery) or non-invasive energy-based techniques (e.g., laser, coblation, ablation, coagulation, microwave energy, radiofrequency, or ultrasound). Finally, the methods and devices herein can be useful for maximizing the tightening effect and reduction in cellulite treatment (and even totally eliminating the cellulite), while minimizing healing time by optimizing tightening (e.g., by controlling the extent of skin pleating, such as by increasing the extent of skin pleating for some applications or skin regions and by decreasing the extent of skin pleating for other applications or skin regions, as described herein).

Furthermore, it is noted that the device and method of the present invention for treatment cellulite, inherently includes 2 advantages: it enables the removal of the cellulite-problem, on the one hand, and the generation of new supportive tissue skeleton (the generation of the crisscross septae).

The device of the present invention is designed to alleviate the appearance of cellulite and/or treating cellulite by tissue engineering, as well as to enhance quality and productivity of skin laxity reduction procedures using advanced robotics, including tissue interference (incision/excision/coring means), machine vision and advanced software.

The device implements skin interference instrument adapted to incise tissue and/or excise tissue and/or core the tissue approach to create new septa like scar tissue which will act as a new septum or septa. The device excises a pattern of predetermined size of dermal septa like scar tissue at desired depth, density, and direction. The performed remaining destructed tissue (and/or holes) in the skin are then stabilized (by wound healing process leading to demarcate the septae-like scar tissue. Such wound healing include hemostasis, inflammation, collagen synthesis and maturation processes) to substantially function as septa.

It is noted that stabilization refers hereinafter to the increase of the yield of collagen synthesis, yielding thicker/denser scar tissue.

Optionally the destructed tissue (and/or holes) are closed using manual compression methods such as compression tape or glue.

According to one embodiment of the present invention, the treatment parameters; i.e., desired density of the interfered tissue, excised tissue, incised tissue, cores, depth, diameter, angle of said at least one robotic arm, orientation of new scar which will act as new septa, etc. are automatically adjusted to the treated patient. Alternatively, the treatment parameters are manually inserted into the device specifically and tailor-made to the treated patient.

The device may include the following elements:
1. At least one Robotic Arm and Controller that control the positioning of the arm relatively to the treated skin area.
2. Skin interference Instrument (and, optionally controls) being selected from either incision/incised tissue and/or excision/excised tissue and/or coring (by means selected from of mechanical means, blades, one or more solid needles, application of temperature to heat and evacuate tissue, application of temperature to heat, application of laser, pulsed electromagnetic field, RF, insertion of threads, application of heat to accelerate collagen synthesis in the tissue, coblation, coagulation, ablation, microwave energy, ultrasound, fractional laser ablation, fractional radiofrequency ablation, coblation, coagulation, microwave energy and/or fractional ultrasonic ablation, application of any other type of energy and any combination thereof).
3. RTC (real time controller) unit that includes at least one engine (e.g., a motor or robotic servo-motor) that controls the rotation, translation as well as the orientation of the robotic arm relatively to the treated skin area; and,
4. Imaging Subsystem—to analyze treatment area and to guide the interference instrument.

According to another embodiment, the device may include vacuum Subsystem-suction is applied to remove, if needed, the excised tissue from the skin following the incision. Or alternatively a retention element (a retainer) is used that will hold the excised tissue, rendering the vacuum subsystem redundant. Hence, a vacuum is thus avoided by such embodiments and rendered unnecessary.

According to another embodiment, the device may include a stretching/compression device (e.g., compression tape) that will enable stretching/compression of the skin, post the interference stage.

### The Interference Means as Coring Means

The skin interference instrument includes e.g., coring punches (e.g., the micro needles); either a single or multi-punch array for either simultaneous or sequentially interfere the skin. It should be noted that the interference instruments could be either single-use (disposable) punches or multi-use (reusable) punches.

According to one embodiment of the present invention, the interference instrument is a mechanical device that allows for small (Diameter of 0.4 to 10.0 mm), circular skin cores to be removed. According to another embodiment of the present invention any cross section of the interference-coring means (other than circular) is also within the scope of the present invention. E.g., circular, rectangular, triangular, hexagonal, oval, staggered rows, parallel rows, a spiral pattern, a square or rectangular pattern, a radial distribution and any combination thereof.

According to one embodiment, the interference-coring instrument, has between 1 and 10 rotating (100-10000 RPM) coring punches that can be set to penetrate the skin surface and core to a depth of 1 to 20 mm.

As disclosed above, suction can be applied to excise the cores from the skin following the incision.

The interference-coring element (e.g., the micro needles) has at least one sharp dermal punch to core out tissue (e.g., 0.25 mm-2.0 mm radius).

According to one embodiment, the dermal punches have a stopping mechanism (a stopper) to limit interference (e.g., coring) depth. A typical interference (e.g., coring) depth is configurable between 1 mm and 10 mm in steps of 0.5 mm.

According to one embodiment, the interference (e.g., coring) depth resolution is +/−0.1 mm.

According to one embodiment, each Individual punch is configured to rotate between 1000-10000 RPM.

According to one embodiment, each individual punch is able to translate into skin up to 3000 mm/sec.

According to one embodiment, each individual punch is configured to rotate at a speed that is less than 30 degree/sec.

According to one embodiment, the puncture angle is normal to the skin (+/−10 deg).

According to one embodiment, the mechanical extraction speed will be 1 cycle per second or faster.

According to one embodiment, the angle of said at least one robotic arm is oriented at an angle in the range of about 0 degrees to about 90 degrees relative to the skin. It should be noted and emphasized that the main aspect of the present invention is to create septa structure that will mimic the male structure (namely, X-shaped). Thus, the angle of penetration into the skin (to excise tissue) will eventually dictate the resulting X-shape septa.

According to one embodiment, if the interference instrument is a coring element, the punch is flushed via saline solution. It should be noted that saline may be used via the punch to flush it between one coring step to the other but also to reduce friction of cored tissue and internal part of the punch during cores evacuation.

The imaging subsystem is provided with illumination means (e.g., emitters such as LEDs) to illuminate the field of view of the imaging subsystem and to keep the cameras of the imaging subsystem exposure time at low latency.

The LED's wavelength is greater than 600 nm (warm white) to enable enough light to be reflected back from skin to cameras. Lower wavelengths tend to get absorbed more by human skin causing dark images.

The treated areas could be any of the body areas e.g., buttocks, lower limbs and abdomen. According to another embodiment, the device of the present invention could be used for focal elimination of redundant dermal tissue for skin tightening, at least partially scar removal etc. It should be noted that according to one embodiment of the present invention, before, during or after the tissue excision (to create new septae-like scar), it could be that at least one of the already existing septa will be at least partially severed.

As disclosed above, post the interference process, the skin is stabilized or tightened together by the stretching/compression device (as discussed hereinbelow) to promote healing thereof per the stretched/compressed tissue. According to one embodiment, the stretching/compression device is adhesive based (e.g., surgical wound closure tape or glue).

According to one embodiment of the present invention, the tensioning of the stretching/compression device, in order for it to effectively stretch the skin, has to be with pulling force of 0 N/mm²-50 N/mm².

It should be noted that according to one embodiment of the present invention, the operator can define in the treatment plan at least one of the following:
assigning surgery area and no-fly zones-where no treatment is provided to said area of skin tissue.
assigning areas with different densities
assigning areas with different interference pattern (e.g., coring pattern, ‘holes’)
assigning the interference instrument's (e.g., punch) depth or different depths;

According to another embodiment of the present invention, adjustment of the treatment parameters could be enabled during treatment, in real-time; either manually, by the operator or automatically, by the system.

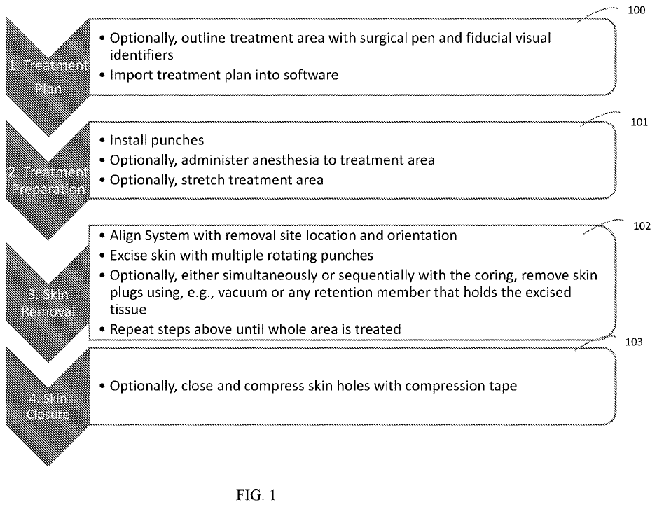

Reference is now being made to FIG. 1 which illustrates the one embodiment of operation of the device of the present invention, utilizing coring means as the interference means.

The first, optional step, step 100, is to outline the skin treatment area, for instance with surgical pen and/or adhesive biocompatible fiducial markers visual identifiers.

An image of treatment area with surgical lines and fiducial markers is enough for treatment planning software to automatically recognize and reconstruct treatment zone in 3D software environment. Thus, Once the treated area is outlined, the treatment plan is finalized (as disclosed hereinafter) and is loaded onto the system.

It should be noted that it is optional that the patient is administered with local anesthesia to avoid any pain during the procedure. According to another embodiment of the present invention, the device additionally comprising at least one injection needle (and/or micro-needle) adapted to administer anesthesia to the treated area.

In case of treatment of folded skin in area to be treated, the operator may stretch the treatment area by applying adhesive stretch tapes. Adhesive tapes (e.g., Tegaderm® or any pressure bandages) put skin under tension by pulling away in preferred directions. It should be noted that it is important to first stretch the skin and only then to excise tissue portions. Otherwise, the skin, due to its flexibility might be caught in the internal area within the interference/drilling/coring means (the punches and/or the needles).

As disclosed above, in some cases (e.g. in case of loose skin), the step of securing said stretching/compression device to said skin region and application of tension (of either stretching or compression) to the skin is performed before said step of said producing a plurality of excised tissue portions in a region of skin tissue. This is to prevent any loosen skin being caught inside the drilling means (punches and/or needles).

According to another embodiment, the stretching/compression device is first stretched or compressed and only thereafter securing the second portion of said stretching/compression device to a different region of said skin.

According to another embodiment, the second portion of said stretching/compression device is secured to a different region of said skin.

The next step, step 101, is to install the punches (and/or the needles) onto the device. The desired punches (and/or the needles) are selected pending the desired density and depth of penetration.

Punches and/or the needles) are sharp, hollow and range from 0.4-4.0 mm in diameter. Larger hole diameters may increase treatment speed but may not be appropriate for all skin types and body areas.

Optionally, a stoper is installed to limit maximum interference depth between 1-10 mm.

Next, step 102, the system is aligned with the area of the skin to be treated. Next, the skin is excised with multiple +/−0.4 to 4 mm (in diameter) punches (or needles).

According to one embodiment, in case the interference instrument is a coring element (punches (or needles), the coring is performed by rotational movement of the punches (or needles), when the same are in contact with the skin. Alternatively, the coring is performed by rotational and translation movement of the punches (or needles). As will be disclosed hereinafter other conventional method to core the tissue (and excise tissue) could be utilized (e.g., RF, laser, ultrasound, microwave etc.). It is within the scope of the present invention where several different modalities will be used to incise/excise the tissue (e.g., mechanical coring, namely the rotational punches, combined with RF energy).

It is also noted that RF (combined with the interference means/drilling/coring punches) will not necessarily be used to excise but to coagulate excision margins-so to accelerate aspects of septae-like scar formation.

Thereafter or simultaneously with the interference means (e.g., coring means, incised tissue means, excised tissue means), the excised tissue can be removed by means of vacuum. It should be noted that the system can utilize interference means (e.g., coring means, incised tissue means, excised tissue means drilling/coring means) that evacuate the skin plugs along with the drilling and, therefore, vacuum means are not needed. In that case at least one retention element, integrated in the drilling means (the punches), is configured to hold the excised tissue (similarly to forceps), rendering the vacuum subsystem redundant. Thus, along with e.g., the drilling/coring of the drilling/coring means (the punches) performed into the skin, the retention element accumulates the excised skin plugs (tissue) and holds it. Thus, there will be no need for application of suction as the suction's main rule is to evacuate the excised skin plugs (tissue). In particular, the at least one retention implement may be implemented as a forceps-like device configured to exert pressure so as to hold the tissue.

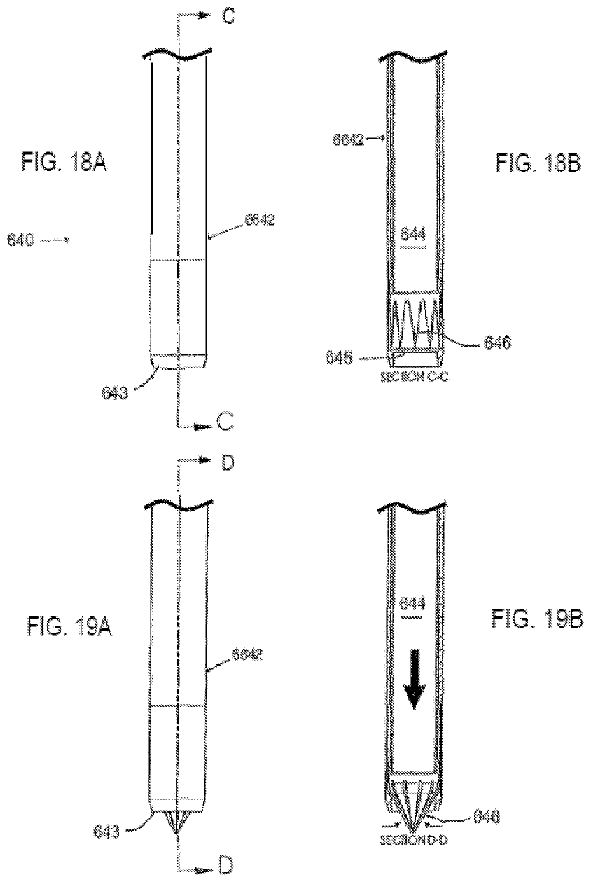

Exemplary implementations of the retention element are shown in FIGS. 18A and 18B, which depict side and longitudinal sectional views, respectively, of a biological unit removal tool having a movable retention member in the form of inner tines in a retracted or undeployed state. FIGS. 19A and 19B show the removal tool in a retention or deployed state. FIGS. 18A, 18B, 19A and 19D are exemplary depictions set forth in U.S. Pat. No. 8,696,686 issued Apr. 15, 2014, the entire contents of which are incorporated herein by reference, including for the apparatuses and methods disclosed therein. The exemplary removal tool 640 of FIGS. 18A, 18B, 19A and 19B has an outer tube or outer member 642 defining a lumen, and an inner tube or inner member 644 with a plurality of movable members or deformable tines 646 mounted on the inner tube. In the retracted position, the deformable tines 646 are flush with the inner diameter of the outer tube 642 and mounted to the distal end of the inner tube 644, which is allowed to move proximal/distal relative to the distal tip 643 of the outer tube. The distal tip 643 has a structure 645 that influences or guides the deformable tines to converge. The structure 645 is configured to assume the form of an inner ridge that guides the tines inward as the inner tube is advanced distally such that the tines converge. Alternatively, the structure may take the form of a taper, a step, an incline or any other form that guides the deformable tines to coapt. In the retention position, at least a portion of the retention member, e.g., the deformable tines, extend beyond the distal tip of the outer elongated member 642. The inner tube with tines may be made of various materials, including shape memory materials, for example, Nitinol, or Elgiloy, or cobalt chromium, or similar material which accommodates repetitive bending without fatigue (or with more tolerant fatigue properties), if desired, at the base of the tines. In some embodiments, the movable retention members need not be in the form of tines, but may be configured as thin wires, filaments, or paddle shaped structures for example, or varying shapes and surface finishes, and of various circumferential distributions.

According to one embodiment, in case the interference means are drilling/coring means (the punches, microneedles), the same generally have a tubular elongated body with a cylindrical profile and a hollow lumen therethrough. According to another embodiment, at least one retention member described herein may be positioned not only at the distal portion of the interference means/drilling/coring means, but also in various locations along the body of the interference means/drilling/coring means, for example, a short distance from the distal end, or midway along the body of the interference means/drilling/coring means, depending upon the configuration of the interference means/drilling/coring means and its intended purpose. The terms “coupled,” or “attached,” or “connected,” or “mounted” as used herein, may mean directly or indirectly coupled, attached, integrated, or mounted through one or more intervening components.

A “retention member” as used herein refers to a structure, or a mechanism, or a number of structures and/or mechanisms that partially or fully retain a biological tissue in a lumen of the drilling means. The retention member may translate into or across the lumen, or radially constrict the lumen in a circumferential manner, for example, simply closing tightly about the tissue, located in the lumen to improve its retention and removal. The retention members described herein may be made of a variety of biocompatible materials, such as polypropylene, polyester, polyurethane, Teflon, Nitinol, stainless steel, etc. The configuration of the retention members may be solid, braided, filamentous, etc., and should not be considered limited to any one particular embodiment.

According to one embodiment the retention member may be movable along an axis of the drilling means (the punches). The retention member may form an integral part of the elongated body or may comprise a separate element attached within the lumen of the elongated body of the drilling means (the punches). In another version, the retention member comprises a portion made of a deformable material and the tool further comprises an actuation device adapted to deform at least the deformable portion of the retention member and constrict a lumen defined therein. For instance, the retention member comprises a plurality of portions made of deformable material, each two being separated by a spacer made of a substantially rigid material, such as Teflon, stainless steel, or titanium. The deformable material may be selected from the group consisting of silicone, rubber, gels, and fluids.

Another aspect of the invention is a biological tissue removal tool (that renders the use of suction redundant) comprising at least one movable retention member in communication with the drilling means (the punches). At least one of the drilling means (the punches) has a lumen sized to receive a biological specimen and a distal tip configured to penetrate a body surface. The retention member moves with respect to the drilling means (the punches) between a retracted position and a retention position in which the retention member is configured to project into or across the drilling means (the punches) proximally to the distal tip so as to impede movement in a distal tip direction of the biological specimen received in the lumen.

The retention member may be located and moveable from outside the drilling means (the punches) into the same. In one embodiment, the retention member is spring-biased, such as torsionally spring-biased, into the retention position. In another form, the retention member slides axially over the drilling means (the punches) between the retracted and retention positions and has a portion that passes into the drilling means (the punches) through an aperture in a wall of the elongated body in the retention position. For instance, the retention member may be a clip having at least two portions passing into the lumen through diametrically opposed apertures in the wall of the drilling means (the punches). In some alternatives, an actuator displaces the retention member between the retracted and retention positions, and the actuator may be automated. The retention member may be rotatable between the retracted and retention positions.

Another example of the at least one movable retention member is as follows. At least a portion of the retention member is axially movable over the drilling means (the punches) and the retention member is radially movable between a retracted position and a retention position, such that in the retention position at least a distal tip of the retention member extends beyond the distal tip of the drilling means (the punches) and converges.

Alternatively, a morcellator-like element could be added. Thus, the cored tissue is grind and then body expels it outwards.

Alternatively, after the interference with the tissue (e.g., the coring), no tissue is extracted.

It should be noted that in case the interference means are e.g., coring means, the coring means could comprise several microneedles (punches) or a single one. It should be further noted that each of which could be independently operated or a sub-group thereof could be operated simultaneously. As stated above, before the coring step, the system aligns at least one of the punches at a predetermined angle relative to the skin. Said angle could be in the range of 0 to 90 (so as to eventually create the crisscross structure).

According to one embodiment there is provided, as the interference means, at least one punch (or needle). The Punches (or needles) could rotate together, or each, individually. According to one embodiment all punches (or needles) are coupled to one common shaft operated by electric DC motor. According to another embodiment, there are multiple shafts operated by

According to one embodiment, the coring rpm is between 1000-10000 rpm.

As disclosed hereinafter, according to one embodiment, the interference means are coring means (e.g., punch/needle) and the dissected skin cores from each punch/needle are pulled up by e.g., vacuum or any retention element(s) e.g., integrated within the punches, into accumulation chamber and eventually through tubing into canister for disposal. To ensure there are no clogs in tubing, liquid (e.g., saline) may be added to the chamber via a dripping mechanism to flush the system from at least one of the punch's end.

The vision subsystem, pointed at where the interference means (e.g., punch) tips will extend, detects 3D location of the skin surface and aligns the interference means (e.g., punch(es)) at a predetermined angle (0 to 90 degrees) relative to the skin plane using moving arm joints. 3D Vision subsystem uses camera(s) and/or infrared laser projector or stereo vision approach for sub millimeter accuracy.

Once aligned, the system translates rotating the interference means (e.g., punch(es)) to patient skin at high speed. Once the interference means (e.g., the punch(es)) approximate the skin they slow-down to a slower speed and then they will penetrate into the skin to 2-10 mm depth.

While inside the skin, the punch(es) use rotation sheer force to fracture and core out skin without compressing skin away from punch tips. Additionally, to avoid unnecessary skin compression, the system uses closed loop force sensor and vision feedback to determine when the punches break tougher epidermis layer and when the punches reach desired depth in dermis.

At the end of the cycle, the system can open vacuum line to pull up and remove dermal tissue core. Next, the punch(es) are pulled back up above skin. Alternatively, the system may include at least one retention element adapted to hold or contain the extract excised tissue (without any applied vacuum).

According to one embodiment, the system can use automation and artificial intelligence algorithms to repeat and deliver described coring procedure according to the treatment plan. It should be noted that the artificial intelligence is used also to determine the treatment plan and coring protocol (e.g., the pattern/density/depth etc. of the coring elements). Such artificial intelligence is used to learn different patients' treatment protocol and outcome thereof; and advise on different aspect upon a new treatment (e.g., where to excise the tissue, at what angle, how many septa to create, how many septa to sever etc.) to deliver appropriate or even better and improved results.

Such use of artificial intelligence results in enablement of fractional septa severing and fractional septa generation methods.

Each coring cycle creates at least 1 hole. Automation arranges and packs the holes patterns to achieve planned density.

By tracking unique fiducial identifiers system remembers where previous holes have been made therefore preventing possibility of overlapped holes. In addition, treatment automation deals with dynamic elements not captured in the treatment plan such as no-go zones, surgical equipment obstructions, bleeding etc.

The final step, is an optional step 103, is the skin closure (e.g., by a compression tape).

According to another embodiment of the present invention, the skin could be stabilized by changing its viscoelastic properties. Such amendments could be achieved by e.g., application of energy or temperature (e.g., freezing the surface thereof by application of substantially reduced temperature thereto). Such freezing will immobilize the skin, resulting in a better, smother and efficient process.

### The Treatment Plan

Before using the device of the present invention, an operator will outline the treatment area on patient's skin. The operator marks treatment area using surgical pen and/or adhesive biocompatible fiducial markers.

An image of treatment area with surgical lines and fiducial markers is enough for treatment planning software to automatically recognize and reconstruct treatment zone in 3D software environment.

Depending on desired density and depth, the operator selects appropriate disposable/multi-use interference means (e.g., punches). It should be noted that according to one embodiment of the present invention, the appropriate disposable/multi-use interference means (e.g., punches) are automatically recommended by the system (based on the treatment parameters; e.g., skin type, lesion to be treated, severity of lesion, desired skin removal density etc.).

The punches (micro needles) are sharp, hollow and range from about +/−0.4-4.0 mm in diameter. Larger hole may increase treatment speed but may not be appropriate for all skin and lesion types. A typical coring depth would be between about 1 to about 10 mm.

The system of the present invention is positioned and orientated over patient skin either by operator manually, or automatically by finding treatment zone using vision subsystem. Vision system registers treatment zone with treatment plan by searching for particular fiducial identifiers or colored lines on the skin.

### The Coring Instruments

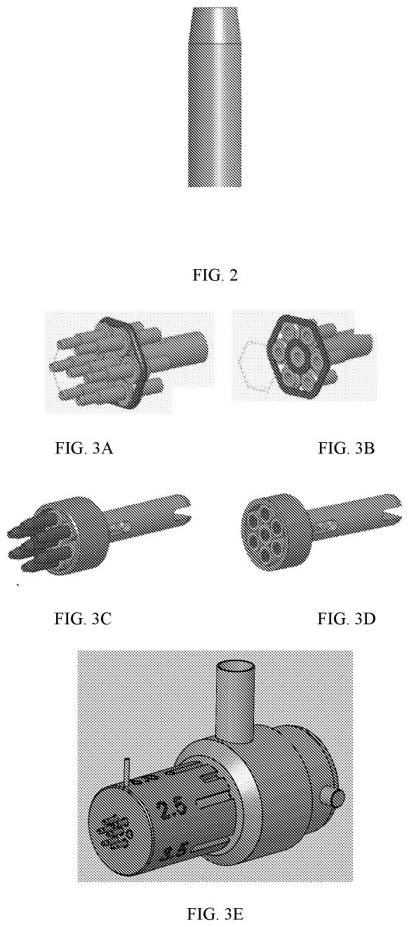

Instrument performs dermal micro-coring process using multiple hollow rotating sharp punches. Each punch, shown on FIG. 2 has cylindrical shape with sharp conical cutting tip at the top. To ensure full dissection each punch has sharp inner edge and outside bevel.

It should be noted that any other cross section area of the punch would work as well.

According to one embodiment of the present invention, there are X simultaneously rotating punches. X is in the range of 3-10.

According to one embodiment, all punches rotate together and coupled to one common shaft operated by electric DC motor. According to another embodiment, each punch rotates individually and may or may not be coupled to one common shaft operated by electric DC motor.

Reference is now made to FIGS. 3A-3D, illustrating the distal end of the applicator have 7 punches, 6 circumferentially provided around a 7th punch. However, it should be emphasized that those figures are an example and any number of punches are applicable.

FIG. 3A-3D illustrate two possible punch rotation drive types: belt driven and friction driven. FIGS. 3A-3B illustrates the belt driven punch rotation type, before and after activation thereof, respectively. FIGS. 3C-3D illustrates the friction driven punch rotation type, before and after activation thereof, respectively.

Reference is now made to FIG. 3E, illustrating another embodiment of the distal end of the applicator have 6 punches (and not 7, as illustrated in FIGS. 3A-3D). As seen in the FIG. 3E, the six micro-coring needles (the punches) are arranged in 2 groups of 3 micro-coring needles, each arranged in vertices of a horizontally laying ‘V’ pattern. Namely, in a pattern of ‘>>’. It should be noted that it is within the scope of the present invention where the six micro-coring needles (the punches) are arranged in at least two horizontally lying ‘V’ shape, oppositely facing. Namely, in a of pattern ‘><’. However, one skilled in the art would appreciate that any pattern could be used. e.g., the pattern of the micro-coring needles (the punches) could be selected from a group consisting of a circular, hexagon, rectangular, square and any combination thereof.

In the figs. the punches are oriented vertically, however the orientation (namely, the angle at which each is positioned relatively to the skin to be treated) is alterable and can vary at a range of about 0 to about 90 degrees.

In some embodiments, the coring RPM is between 1000-10000 RPM. Punches can translate together back and forth on a leadscrew or using robotic arm itself.

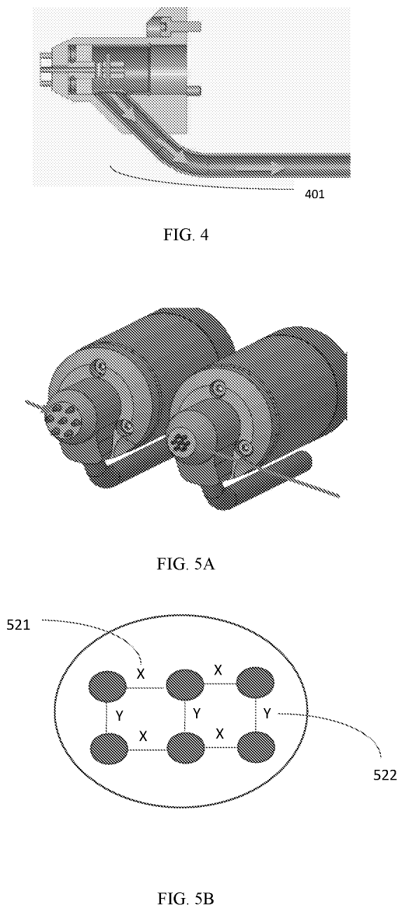

The punches are connected to skin core accumulation chamber. Dissected skin cores from each punch are pulled up by e.g., vacuum (see arrows 401) into accumulation chamber and eventually through tubing into canister (not shown) for disposal (see FIG. 4). It is noted that, as an alternative to the vacuum, the system may include at least one retention element adapted to hold or contain the extract excised tissue (without any applied vacuum). To ensure there are no clogs in tubing, liquid (e.g., saline) may be added to the chamber via a dripping mechanism to flush the system.

According to another embodiment, the liquid (e.g., saline) is added to reduce friction during the coring step.

According to one embodiment of the present invention, only one arm with 1 or more punch is utilized in the system. According to another embodiment of the present invention, more than one arm, each of which utilizes 1 or more punches is embodied in the system (as illustrated in FIG. 5A). In such an embodiment, each arm could utilize 1 or more punches with the same properties (width, depth, cross section etc.) or alternatively, each arm would enclose one or more punch(es), each (or all) with individual/distinct properties.

According to another embodiment, each arm (and punches thereof) is characterized by different properties (e.g., width, depth, orientation cross section of the punches, translation speed, rotation speed etc.).

According to another embodiment, all arms may include the same mechanism; alternatively, each arm comprises a different mechanism, e.g., different incision/excision means ((e.g., one arm makes an incision and the second arm is used for seeding or insertion/injection of additives, as disclosed hereinafter (e.g., threads, hyaluronic acid etc.)).

According to another embodiment of the present invention each punch is activated independently, such that in some embodiments, in the at least one arm of the device, there are several punches. However, each would be operated individually; thus, the operator may activate only a few of the punches and not all.

According to another embodiment of the present invention, the distance between each punch could be adjusted. Ref. is now made to FIG. 5B, which illustrates one arm 510 of the device having 6 punches 520, space apart at a distance X (see numerical ref. 521) and Y (see numerical ref. 522) from each other. According to one embodiment, said X and Y are adjustable such that the distances between the punches are changeable to better adjust thereof to the treatment.

As discussed above, the coring element is used to facilitate the generation of septa but also could be used to at least partially sever at least one septum.

### Automation and Artificial Intelligence Algorithms

According to one embodiment, the system uses automation and artificial intelligence algorithms to analyze the mechanical visualization input and to determine and establish the most appropriate coring pattern (orientation, density, amount etc.) and plan (e.g., where to excise first, where to sever first etc.). Thereafter, the artificial intelligence instructs to repeat and deliver described coring procedure according to the treatment plan rules.

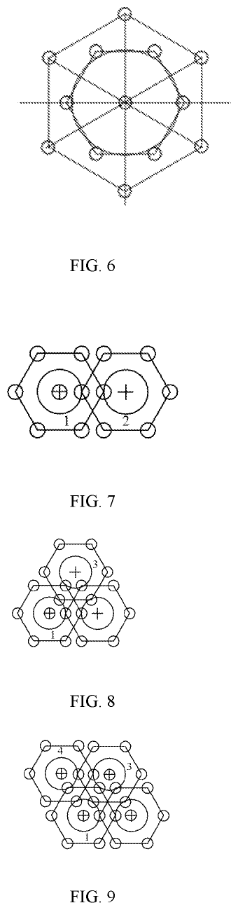

Automation arranges and packs hex patterns to achieve planned density. For example, on FIGS. 7-9, one instrument design may spread out punches allowing overlapping patterns, while another design may have punches packed tightly together. By tracking unique fiducial identifiers system remembers where previous holes have been made therefore preventing possibility of overlapped holes. In addition, treatment automation deals with dynamic elements not captured in the treatment plan such as no-go zones, surgical equipment obstructions, bleeding etc.

According to one embodiment, the overlapping patterns could have at least one point of excised tissue portion.

According to another embodiment of the present invention, the system utilizes artificial intelligence and/or mechanical visualization, OCT, Ultrasound, machine learning algorithms and/or image processing to provide inform decision as to the coring location. In other words, the system first scans the tissue to be treated and by means of at least one selected from a group consisting of artificial intelligence, mechanical visualization, OCT, Ultrasound, machine learning algorithms, image processing and any combination thereof, the system decides where it would be most beneficial to perform the coring.

Septa Generation and, Optionally, Septa Severing

As disclosed above, the main object of the present invention is to provide an effective cellulite treatment by mimicking the male septa structure. Namely, the crisscross structure of the septa. Thus, according to a preferred embodiment of the present invention, the coring element will excise tissue and thereby create septae-like scar tissue (that may be stabilized thereafter by means, e.g., RF energy). The septa structure will resemble crisscross (X-shaped) structure that mimics the male's septa structure.

Such generation of septae-like scar tissue is enabled by the interference means. Such means are selected from a group consisting of either incision/incised tissue and/or excision/excised tissue and/or coring (by means selected from of mechanical means, blades, one or more solid needles, application of temperature to heat and evacuate tissue, application of temperature to heat, application of laser, insertion of threads, pulsed electromagnetic field, RF, coblation, coagulation, ablation, microwave energy, ultrasound, application of heat to accelerate collagen synthesis in the tissue, fractional laser ablation, fractional radiofrequency ablation, coblation, coagulation, microwave energy and/or fractional ultrasonic ablation, application of any other type of energy and any combination thereof).

In such an embodiment, the device will orient the interference means is oriented at an angle A relative to the treated skin area (e.g., the buttocks, lower limbs and abdomen). Angle A is in the range of about 0 to about 90 degrees relative to the skin. It is noted that the angle will determine the resultant crisscross shape of the generated septa.

According to another embodiment, the interference means will not only create septa (by tissue excision) but will also at least partially sever at least one septum. According to one embodiment, the excision of tissue (to either sever septa or generate septa) is performed by means selected from mechanical translatable/rotatable punches, RF energy, laser (or any other optical means), ultrasound, microwave energy and any combination thereof.

### Stabilization of the Generated Septae

It is within the scope of the present invention that the interference procedure (and/or the scar generation) trigger wound healing process that result in generation of new septa.
such process will, according to one embodiment, require stabilization to ensure the same remains.

According to one embodiment such stabilization means could be e.g., coagulation (by application of RF energy).

According to another embodiment of the present invention, stabilization of the crisscross (X-shaped) septa structure could be provided by threading a suture thread to anchor the septa to their location (from the dermis and to the fascia).

According to another embodiment of the present invention, stabilization of the crisscross (X-shaped) septa structure could be provided by threading a suture thread horizontally (parallel the skin) to anchor the septa to their location.

Thus, the process of alleviating appearance of cellulite will includes steps of
(a) scanning the treated area using an over-skin 3D imaging module.
(b) identifying and mapping the Septa matrix.
(c) Analyzing (using image processing and artificial intelligence) which Septa to cut and where to generate new ones.
(d) cutting the selected Septa (by the coring element), and
(e) generating new Septa between dermis and up to fascia depth, in an X-shaped structure guided by imaging (e.g., under-skin imaging module).

### Directional Tightening—Optional Added Treatment

At the end of the treatment, the operator can use a stretching/compression device to close holes in the skin and promote healing per the new dimensions of the cored area, as employed by e.g., the compression.

According to one embodiment of the present invention, the stretching/compression device is an elastic compression tapes to close holes in the skin. Compressing skin together enables wound healing and collagen accumulation and adherence of the cored walls per its modified (compressed) configuration. Accordingly, with compression, cored holes are not as circles anymore, but ellipsoid and configured to be stabilized by new collagen in that position, promoting healing with the result of aesthetic skin tightening results due to the accumulated compressed cores per axis (with less chance of scars). Such tightening will result in lifting and stabilization of sagging skin.

The stretching/compression device disclosed herein creates compression on the internal area and tension on the external area and eliminates unwanted puncture scars.

According to one embodiment of the present invention, the tension applied can be adjusted based on skin type to produce best results.

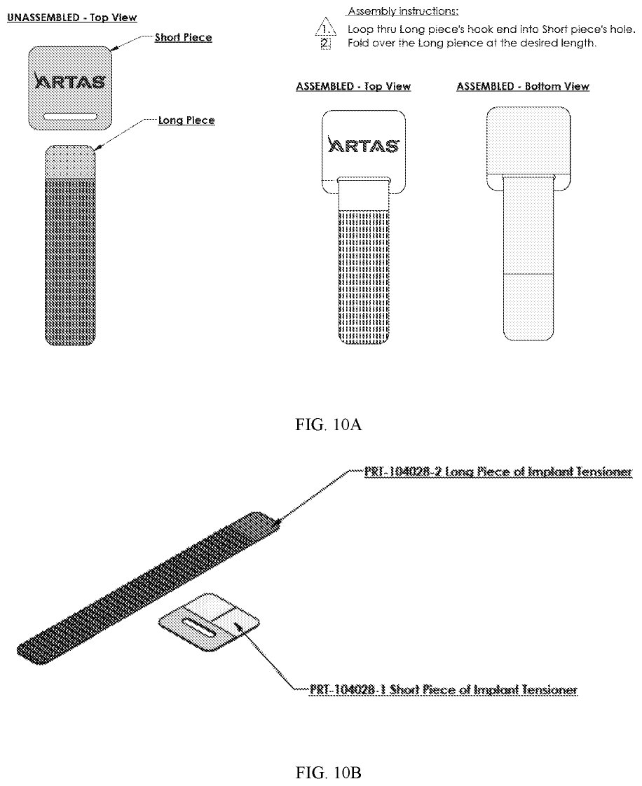

Reference is now made to FIGS. 10A-10B illustrating one embodiment of the stretching/compression device.

According to this embodiment of the present invention, the stretching/compression device has a long and short portion. The short portion comprises at least one buckle-like element having at least one slot hole therewithin. The long portion is adapted to be connected to the short side through said at least one slot hole therewithin. The long portion is threaded through said slot and secured to the short portion (as detailed hereinbelow). Said securement of said long portion to said short portion is by means of attaching at least one adhesive layer in said long portion to at least one adhesive layer in said short portion.

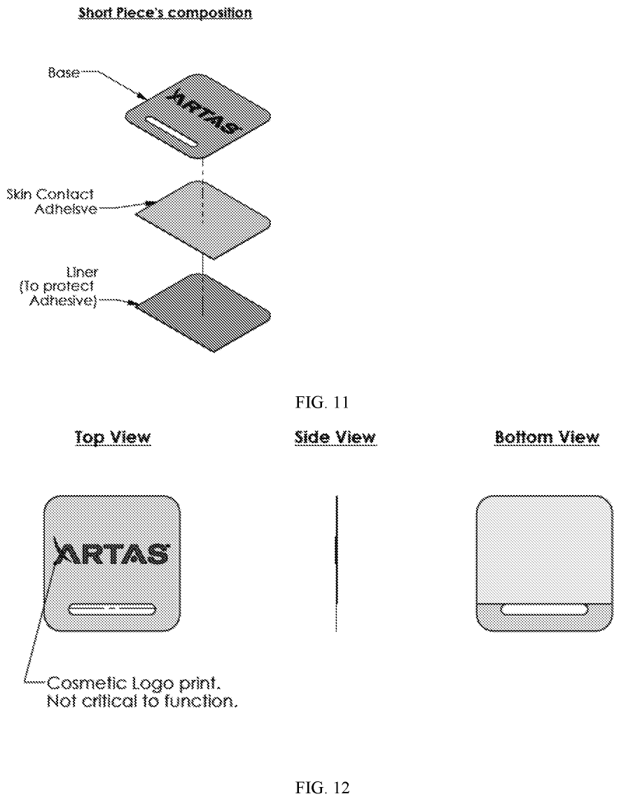

Reference is now made to FIGS. 11-12 illustrating the short side, according to this embodiment, of the stretching/compression device. According to this embodiment, the short side has base, adhesive, and liner.

The base can be made from any material that is strong enough to withstand, for example, 10 PSI in shear force.

The adhesive can be made from any material that is strong enough to withstand, for example, 10 PSI in shear force and the adhesive should adhere to skin well.

The liner is a cover that protects the adhesive until it is to be used.

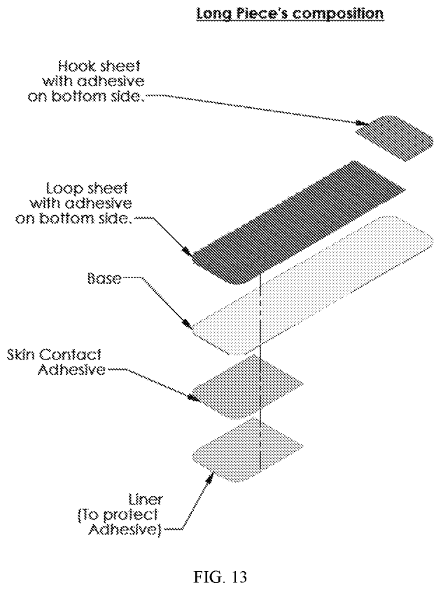

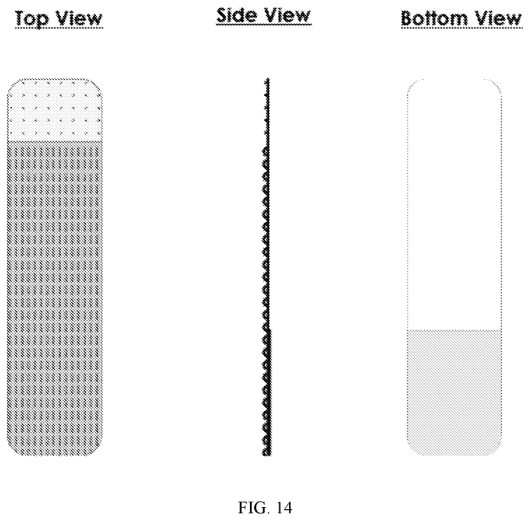

Reference is now made to FIGS. 13-14 illustrating the long side, according to this embodiment, of the stretching/compression device.

According to this embodiment, the long side has a base, an adhesive, a liner, and hook & loop sheets.

The base can be made from any material that is strong enough to withstand, for example, 10 PSI in shear force.

The adhesive can be made from any material that is strong enough to withstand, for example, 10 PSI in shear force and it should adhere to skin well.

The liner is a cover that protects the adhesive until it is to be used.

According to one embodiment, the hook and loop component (e.g., sheet) is Velcro. In an example, the hook sheet is the male side where it has tiny semi-rigid hooks on the top side and the loop sheet is the female side where it has thin loops on the top side. When the hook top side and loop top side come in contact with each other, the hooks hook onto the loops.

On the bottom side of both sheets, there is adhesive to allow the sheets to adhere to the base. This is not always necessary. An alternative is that the sheet acts as the base layer if the sheet is strong enough.

According to one embodiment, the loop sheet covers most of the long piece interface. This allows for smooth tape movement since the loop sheet may be thinner than the hook sheet. It is possible to reverse this; the hook sheet covers most of the long piece, but the hook sheet should be thin enough to be flexible enough to fold over (see side view note).

Once the stretching/compression device is placed over the holes in the skin, the operator stretches the same to create compression and/or tension to the desired level. Once the desired tension level is reached, the stretching/compression device can be closed and secured.

The application of the stretching/compression device will result in direction tightening of the skin.

The directionality of the skin region to which the stretching/compression device is applied, can also be optimized. In particular embodiments, the direction of skin tightening is determined by the directionality of the tensile force or compressive force being applied. It can be in the x-, y-, and/or z-direction with respect to the device or skin region.

The optimization of the applied tension of the stretching/compression device can provide numerous benefits. For instance, such tunability can allow real-time control of compressing and/or expanding the stretching/compression device after affixation thereof to the skin. This level of control can allow for personalized treatment of the patient based on the disease, disorder, or condition to be treated; the optimal cosmetic effect to be achieved; the optimal closure process to be achieved; and/or the timing and extent of the healing process observed for the particular patient. Furthermore, tunability can allow for less discriminate control over how the incisions or excisions in the skin region are made, as well as more discriminate control over selectively closing or opening the incisions or excisions.

The stretching/compression device can be affixed to the entire treated skin region or in a portion of the treated skin region. Directional or non-directional tightening can be achieved by producing a geometric arrangement of incisions and/or excisions that are treated similarly. Alternatively, such tightening can be achieved by a non-geometric arrangement of incisions and/or excisions in which only some of the incisions and/or excisions are opened or closed using the stretching/compression device.

It should be noted that when incision or excision occur—then wound healing process starts and, as commonly known, includes collagen synthesis and maturation. Thus, it is within the core of the present invention to facilitate its construction and accumulation per deformed cored area(s).

The tunable dressing can include an adhesive layer (e.g., formed from any adhesive material described herein). The adhesive layer can be continuous (i.e., a continuous layer of one or more adhesive materials attached to the proximal surface of a dressing) or discontinuous (i.e., a non-continuous layer of one or more adhesive materials attached to the proximal surface of a dressing). The adhesive layer can include any useful arrangement of the adhesive material. For instance, the adhesive layer can be tunable and allows for controlled compression or expansion. In some embodiments, an adhesive layer includes a random, non-geometric, or geometric array of an adhesive material for tunability. In particular embodiments, the array allows for directional or non-directional compression and/or expansion as the dressing compresses and/or expands. In particular embodiments, the adhesive layer is discontinuous and includes an array of an adhesive material (e.g., an array of dots, where each dot gets closer together as the dressing compresses and each dot gets further apart as the dressing expands). Exemplary adhesive materials are described herein and include materials that promote collagen cross-linking, such as riboflavin or Rose Bengal, synthetic glues (e.g., cyanoacrylate, polyethylene glycol, or gelatin-resorcinol-formaldehyde), or biologic sealants (e.g., albumin-based or fibrin-based sealants that promote clotting).

The stretching/compression device can also include at least one occlusion layer (e.g., to control humidity and/or promote wound healing), at least one absorption layer (e.g., to absorb wound exudate), at least one reinforcement layer (e.g., to reinforce the layer and optionally formed from low-density polyethylene (LDPE), fluorinated ethylene propylene (FEP), or nylon), and/or at least one delivery layer (e.g., to delivery one or more therapeutic agents to enhance treatment thereof).

The stretching/compression device can be of any cosmetically appealing color, shape, and/or material. For example, the stretching/compression device can be provided in a skin tone color or is transparent or semi-transparent. Such transparent or semi-transparent dressings can additionally be helpful for visualization, e.g., for real-time tunability of the dressing and/or for affixing the stretching/compression device to the treated skin region.

According to another embodiment of the present invention, the stretching/compression device could either first be applied (i.e., secured) to skin (post excision of the skin portion) and only thereafter application of tension forces are applied thereto to provide the directional tightening of the skin.

According to another embodiment of the present invention, the stretching/compression device could either first be stretched and only then applied (i.e., secured) to skin (post excision of the skin portion). Once applied when the same is stretched the stretching/compression device (as it is an elastic dressing) would compress back to its original shape and hance apply compression tension to the skin thereto to provide the directional tightening of the skin.

In other words, the stretching/compression device could first go through a pretreatment, where stretching forces are applied thereto (for example by means of a dedicated device) and, once it is fully/partially stretched it is applied to the skin.

According to another embodiment of the present invention, the stretching/compression device that could be employed is simply an adhesive tape, e.g., 3M™ Tegaderm™, HP Transparent Film Dressing (see https://www.3m.com/3M/en_US/company-us/all-3m-products/~/3M-Tegaderm-HP-Transparent-Film-Dressing/?N=5002385+3293321973&rt=rud).

The present invention relates to various methods and devices (e.g., the stretching/compression device) used to selectively open or close incisions and/or excisions (e.g., all or a portion of such incisions, such as microslits, and/or excisions, such as holes) formed in the skin region by the incised or excised tissue portions. The devices can be affixed to the entire treated skin region or in a portion of the treated skin region, which allow for directional or non-directional tightening by producing a geometric or non-geometric arrangement of incisions and/or excisions that are treated similarly or differently. Further, the devices can provide uniform or non-uniform compression and/or expression across the entire device or a portion thereof. Accordingly, these methods and devices can result in reducing the skin surface and/or tightening of the skin.

The methods can include contraction or expansion in one or more directions in at least a portion of the device (e.g., the dressing). The methods include, for example, affixing the stretching/compression device to a skin region having a plurality of incised tissue portions and/or excised tissue portions (e.g., where at least two of said tissue portions has at least one dimension that is less than about 1 mm or an areal dimension that is less than about 1 mm²). The device provides contraction or expansion of the skin region in one or more directions (e.g., in the x-, y-, z-, xy-, xz-, yz-, and/or xyz-directions, as described herein), where such contraction or expansion can be uniform or non-uniform. Furthermore, contraction or expansion arises by exposing an affixed device to one or more external stimuli (e.g., any described herein) that results of application of force (e.g., compression or stretching forces) on the stretching/compression device. In addition, such contraction and/or expansion can be adjusted after affixing the device. For example, after treating the skin and affixing the device, the device can be further expanded or to compress the skin region. In this manner, the device is tunable/adjustable.

The present invention also includes methods of tightening skin in a preferred direction.

The present invention also includes optimizing the dimension of the incised or excised tissue portions to promote wound healing. Exemplary dimensions include circular and non-circular holes, such as elliptical holes. Non-circular holes can be formed by using an apparatus having a non-circular cross-section (e.g., a blade or a tube, such as a hollow tube, having a non-circular cross-section) or by pre-stretching the skin before treatment with an apparatus having a circular cross-section (e.g., a circular coring needle generates an elliptical hole in a non-stretched skin). In some embodiments, the long axis of the ellipse is perpendicular to the pre-stretching direction, where the elliptical hole can generate skin tightening preferentially in the direction of the short axis of the ellipse. Accordingly, the stretching/compression device can be affixed to a skin portion including one or more holes or one or more incised or excised tissue portions having one or more geometries.

It should be noted that when incision or excision occur-then wound healing process starts and, as commonly known, includes collagen synthesis and maturation. Thus, it is within the core of the present invention to facilitate its construction and accumulation per deformed cored area(s).

### Adhesive Materials That Can Be Integrated in the Stretching/compression Device.

An adhesive can be used within the dressing (e.g., as in the adhesive layer) or used in combination with any method described herein to promote skin tightening.

The adhesive can be a pressure-sensitive adhesive (PSA). The properties of pressure sensitive adhesives are governed by three parameters, tack (initial adhesion), peel strength (adhesion), and shear strength (cohesion). Pressure-sensitive adhesives can be synthesized in several ways, including solvent-borne, water-borne, and hot-melt methods. Tack is the initial adhesion under slight pressure and short dwell time and depends on the adhesive's ability to wet the contact surface. Peel strength is the force required to remove the PSA from the contact surface. The peel adhesion depends on many factors, including the tack, bonding history (e.g. force, dwell time), and adhesive composition. Shear strength is a measure of the adhesive's resistance to continuous stress. The shear strength is influenced by several parameters, including internal adhesion, cross-linking, and viscoelastic properties of the adhesive. Permanent adhesives are generally resistant to debonding and possess very high peel and shear strength.

Exemplary adhesives include a biocompatible matrix (e.g., those including at least one of collagen (e.g., a collagen sponge), low melting agarose (LMA), polylactic acid (PLA), and/or hyaluronic acid (e.g., hyaluranon); a photosensitizer (e.g., Rose Bengal, riboflavin-5-phosphate (R-5-P), methylene blue (MB), N-hydroxypyridine-2-(1H)-thione (N-HTP), a porphyrin, or a chlorin, as well as precursors thereof); a photochemical agent (e.g., 1,8 naphthalimide); a synthetic glue (e.g., a cyanoacrylate adhesive, a polyethylene glycol adhesive, or a gelatin-resorcinol-formaldehyde adhesive); or a biologic sealant (e.g., a mixture of riboflavin-5-phosphate and fibrinogen, a fibrin-based sealant, an albumin-based sealant, or a starch-based sealant). In particular embodiments, the adhesive is biodegradable.

Exemplary pressure-sensitive adhesives include natural rubber, synthetic rubber (e.g., styrene-butadiene and styrene-ethylene copolymers), polyvinyl ether, polyurethane, acrylic, silicones, and ethylene-vinyl acetate copolymers. A copolymer's adhesive properties can be altered by varying the composition (via monomer components) changing the glass transition temperature (Tg) or degree of cross-linking. In general, a copolymer with a lower Tg is less rigid and a copolymer with a higher Tg is more rigid. The tack of PSAs can be altered by the addition of components to alter the viscosity or mechanical properties. Exemplary pressure sensitive adhesives are described in Czech et al., “Pressure-Sensitive Adhesives for Medical Applications,” in Wide Spectra of Quality Control, Dr. Isin Akyar (Ed., published by InTech), Chapter 17(2011 ), which is hereby incorporated by reference in its entirety.

In one exemplary technique, a photosensitizer is applied to the tissue (e.g., Rose Bengal (RB) at concentration of less than 1.0% weight per volume in a buffer, e.g., phosphate buffered saline to form a skin tissue-RB complex), and then the tissue is irradiated with electromagnetic energy to produce a seal (e.g., irradiated at a wavelength of at least 488, at less than 2000J/cm<2>, and/or at less than 1.5 W/cm<2>, e.g., about 0.6 W/cm<2>). This exemplary technique is described in U.S. Pat. No. 7,073,510, which is incorporated by reference in its entirety. In another exemplary technique, a laser can be used for tissue welding. In yet another exemplary technique, a photochemical agent is applied to the tissue, and then the tissue is irradiated with visible light to produce a seal.

According to one embodiment of the present invention, therapeutic agents can be integrated within the stretching/compression device to be released to the skin's holes to accelerate healing thereof. Exemplary agents include one or more growth factors (e.g., vascular endothelial growth factor (VEGF), platelet-derived growth factor (PDGF), transforming growth factor beta (TGF-β), fibroblast growth factor (FGF), epidermal growth factor (EGF), and keratinocyte growth factor); one or more stem cells (e.g., adipose tissue-derived stem cells and/or bone marrow-derived mesenchymal stem cells); steroids (for example, steroids to prevent edema), agents which prevent post-inflammatory skin hyperpigmentation (e.g., hydroquinone, azelaic acid, kojic acid, mandelic acid, or niacinamide); one or more analgesics (e.g., paracetamol/acetaminophen, aspirin, a non-steroidal anti-inflammatory drug, as described herein, a cyclooxygenase-2-specific inhibitor, as described herein, dextropropoxyphene, co-codamol, an opioid (e.g., morphine, codeine, oxycodone, hydrocodone, dihydromorphine, pethidine, buprenorphine, tramadol, or methadone), fentanyl, procaine, lidocaine, tetracaine, dibucaine, benzocaine, p-butylaminobenzoic acid 2-(diethylamino) ethyl ester HCl, mepivacaine, piperocaine, dyclonine, or venlafaxine); one or more antibiotics (e.g., cephalosporin, bactitracin, polymyxin B sulfate, neomycin, bismuth tribromophenate, or polysporin); one or more antifungals (e.g., nystatin); one or more anti-inflammatory agents (e.g., a non-steroidal anti-inflammatory drug (NSAID, e.g., ibuprofen, ketoprofen, flurbiprofen, piroxicam, indomethacin, diclofenac, sulindac, naproxen, aspirin, ketorolac, or tacrolimus), a cyclooxygenase-2-specific inhibitor (COX-2 inhibitor, e.g., rofecoxib (Vioxx®), etoricoxib, and celecoxib (Celebrex®)), a glucocorticoid agent, a specific cytokine directed at T lymphocyte function), a steroid (e.g., a corticosteroid, such as a glucocorticoid (e.g., aldosterone, beclometasone, betamethasone, cortisone, deoxycorticosterone acetate, dexamethasone, fludrocortisone acetate, hydrocortisone, methylprednisolone, prednisone, prednisolone, or triamcinolone) or a mineralocorticoid agent (e.g., aldosterone, corticosterone, or deoxycorticosterone)), or an immune selective anti-inflammatory derivative (e.g., phenylalanine-glutamine-glycine (FEG) and its D-isomeric form (feG))); one or more antimicrobials (e.g., chlorhexidine gluconate, iodine (e.g., tincture of iodine, povidone-iodine, or Lugol's iodine), or silver, such as silver nitrate (e.g., as a 0.5% solution), silver sulfadiazine (e.g., as a cream), or Ag<+>in one or more useful carriers (e.g., an alginate, such as Acticoat® including nanocrystalline silver coating in high density polyethylene, available from Smith & Nephew, London, U.K., or Silvercel® including a mixture of alginate, carboxymethylcellulose, and silver coated nylon fibers, available from Systagenix, Gatwick, U.K. ; a foam (e.g., Contreet® Foam including a soft hydrophilic polyurethane foam and silver, available from Coloplast A/S, Humlebaek, Denmark); a hydrocolloid (e.g., Aquacel® Ag including ionic silver and a hydrocolloid, available from Conva Tec Inc., Skillman, N.J.); or a hydrogel (e.g., Silvasorb® including ionic silver, available from Medline Industries Inc., Mansfield, Mass.)); one or more antiseptics (e.g., an alcohol, such as ethanol (e.g., 60-90%), 1-propanol (e.g., 60-70%), as well as mixtures of 2-propanol/isopropanol; boric acid; calcium hypochlorite; hydrogen peroxide; manuka honey and/or methylglyoxal; a phenol (carbolic acid) compound, e.g., sodium 3,5-dibromo-4-hydroxybenzene sulfonate, trichlorophenylmethyl iodosalicyl, or triclosan; a polyhexanide compound, e.g., polyhexamethylene biguanide (PHMB); a quaternary ammonium compound, such as benzalkonium chloride (BAC), benzethonium chloride (BZT), cetyl trimethylammonium bromide (CTMB), cetylpyridinium chloride (CPC), chlorhexidine (e.g., chlorhexidine gluconate), or octenidine (e.g., octenidine dihydrochloride); sodium bicarbonate; sodium chloride; sodium hypochlorite (e.g., optionally in combination with boric acid in Dakin's solution); or a triarylmethane dye (e.g., Brilliant Green)); one or more antiproliferative agents (e.g., sirolimus, tacrolimus, zotarolimus, biolimus, or paclitaxel); one or more emollients; one or more hemostatic agents (e.g., collagen, such as microfibrillar collagen, chitosan, calcium-loaded zeolite, cellulose, anhydrous aluminum sulfate, silver nitrate, potassium alum, titanium oxide, fibrinogen, epinephrine, calcium alginate, poly-N-acetyl glucosamine, thrombin, coagulation factor(s) (e.g., II, V, VII, VIII, IX, X, XI, XIII, or Von Willebrand factor, as well as activated forms thereof), a procoagulant (e.g., propyl gallate), an anti-fibrinolytic agent (e.g., epsilon aminocaproic acid or tranexamic acid), and the like); one or more procoagulative agents (e.g., any hemostatic agent described herein, desmopressin, coagulation factor(s) (e.g., II, V, VII, VIII, IX, X, XI, XIII, or Von Willebrand factor, as well as activated forms thereof), procoagulants (e.g., propyl gallate), antifibrinolytics (e.g., epsilon aminocaproic acid), and the like); one or more anticoagulative agents (e.g., heparin or derivatives thereof, such as low molecular weight heparin, fondaparinux, or idraparinux; an anti-platelet agent, such as aspirin, dipyridamole, ticlopidine, clopidogrel, or prasugrel; a factor Xa inhibitor, such as a direct factor Xa inhibitor, e.g., apixaban or rivaroxaban; a thrombin inhibitor, such as a direct thrombin inhibitor, e.g., argatroban, bivalirudin, dabigatran, hirudin, lepirudin, or ximelagatran; or a coumarin derivative or vitamin K antagonist, such as warfarin (coumadin), acenocoumarol, atromentin, phenindione, or phenprocoumon); one or more immune modulators, including corticosteroids and non-steroidal immune modulators (e.g., NSAIDS, such as any described herein); one or more proteins; or one or more vitamins (e.g., vitamin A, C, and/or E).

For the skin tightening methods described herein, the use of anticoagulative and/or procoagulative agents may be of particular relevance. For instance, by controlling the extent of bleeding and/or clotting in the incisions and/or excisions, the skin tightening effect can be more effectively controlled. Thus, in some embodiments, the methods and devices herein include one or more anticoagulative agents, one or more procoagulative agents, one or more hemostatic agents, or combinations thereof. In particular embodiments, the therapeutic agent controls the extent of bleeding and/or clotting in the treated skin region, including the use one or more anticoagulative agents (e.g., to inhibit clot formation prior to skin healing or slit/hole closure) and/or one or more hemostatic or procoagulative agents.

### Methods for Treating Skin Regions

The present invention relates to methods and devices that can be applied to treated skin regions. In particular embodiments, these regions are treated with one or more procedures to improve skin appearance. Accordingly, the stretching/compression device, and methods herein can be useful for skin rejuvenation (e.g., removal of pigment, tattoo removal, veins (e.g., spider veins or reticular veins), and/or vessels in the skin) or for treating acne, allodynia, blemishes, ectopic dermatitis, hyperpigmentation, hyperplasia (e.g., lentigo or keratosis), loss of translucency, loss of elasticity, melasma (e.g., epidermal, dermal, or mixed subtypes), photodamage, rashes (e.g., erythematous, macular, papular, and/or bullous conditions), psoriasis, rhytides (or wrinkles, e.g., crow's feet, age-related rhytides, sun-related rhytides, or heredity-related rhytides), sallow color, scar contracture (e.g., relaxation of scar tissue), scarring (e.g., due to acne, surgery, or other trauma), skin aging, skin contraction (e.g., excessive tension in the skin), skin irritation/sensitivity, skin laxity (e.g., loose or sagging skin or other skin irregularities), striae (or stretch marks), vascular lesions (e.g., angioma, erythema, hemangioma, papule, port wine stain, rosacea, reticular vein, or telangiectasia), or any other unwanted skin irregularities.

Such treatments can be included any parts of the body, including the face (e.g., eyelid, cheeks, chin, forehead, lips, or nose), neck, thighs, chest (e.g., as in a breast lift), arms, legs, nose, forehead, buttocks, and/or back. Accordingly, the devices on the invention can be arranged or configured to be amenable to the size or geometry of different body regions. Such arrangements and configurations can include any useful shape (e.g., linear, curved, or stellate), size, and/or depth.

In some embodiments, the incised or excised tissue portions forms at least one interference (e.g., hole) in the skin region, where the diameter or width of the hole is less than about 1.0 mm and results in a tissue portion having a diameter or width that is less than about 2.0 mm. In further embodiments, the tissue portion has a diameter or width that is less than about 2.0 mm and a length of more than about 1.0 mm. In particular embodiments, relatively small dimensions of the tissue portions can promote healing while minimizing the formation of scars. Furthermore, the fractional treatment resulting in a plurality of tissue portions can be incised or excised in any beneficial pattern within the skin region. Exemplary patterns within the skin region include tile patterns or fractal-like shapes, where the array of hollow tubes can be arranged, e.g., in a base, to effectuate such a pattern (see FIGS. 7-9). It should be emphasized that according to one embodiment of the present invention, there can be an overlap between one coring step to the other (by vertex or facet); and, according to another embodiment of the present invention there can an overlap in the cross section between consecutive coring steps (as can be seen in FIG. 7). In other words, the first cross section area of the first coring step is, as shown, e.g., in FIG. 7, is hexagonal. The next step, according to one embodiment of the present invention, could provide coring in any location within said hexagonal cross section of the first step.

According to another embodiment of the present invention, a higher density and/or smaller spacing of tissue portions (e.g., slits and/or holes) can be incised or excised in the skin in the center of the pattern or in thicker portions of the skin. In another example, the pattern within the skin can be random, staggered rows, parallel rows, a circular pattern, a spiral pattern, a square or rectangular pattern, a triangular pattern, a hexagonal pattern, a radial distribution, or a combination of one or more such patterns of the incised or excised tissue portions. The pattern can arise from modifications to the average length, depth, or width of an incised or excised tissue portion, as well as the density, orientation, and spacing between such incisions and/or excisions (e.g., by using an apparatus having one or more blades or tubes with differing lengths, widths, or geometries that are arranged in a particular density or spacing pattern). Such patterns can be optimized to promote unidirectional, non-directional, or multidirectional contraction or expansion of skin (e.g., in the x-direction, y-direction, x-direction, x-y plane, y-z plane, x-z plane, and/or xyz-plane), such as by modifying the average length, depth, width, density, orientation, and/or spacing between incisions and/or excisions.

Any useful portion of the skin can be incised or excised. Such tissue portions can include epidermal tissue, dermal tissue, and/or cells or tissue proximal to the dermal/fatty layer boundary (e.g., stem cells).

According to another embodiment of the present invention, the interference (e.g., holes) in the tissue could be achieved by using a scalpel, application of energy (e.g., laser), coblation, ablation, coagulation, ultrasound, microwave energy, RF, application of heat (to evaporate skin portions), mechanical applicator that ‘drills’ through the skin whilst suction is applies (during the drilling or thereafter) to removes the excised skin portion, or any another instrument. For example, an excision includes any removed tissue or tissue portion from a skin region, which can result in excised tissue portions having a particular geometry (e.g., a cylindrical geometry, rectangular, triangle etc. or any arbitrary shape) and produce one or more holes (i.e., negative space created by the removal of tissue) in the skin region. Exemplary methods of forming excised tissue portions or excisions include use of one or more hollow needles (optionally include one or more notches, extensions, protrusions, and/or barbs), one or more microaugers, one or more microabraders, any useful tool for forming excisions, or any methods and apparatuses described herein.

### Safety Subsystem

According to one embodiment of the present invention, the following safety issues are taken into account.
Emergency Power Off switch that immediately removes all energy and motions from the system
all operative robotic arms stopes and descends slowly to rest in case of total power loss
Needles/Punches are automatically retracted to safe location within mechanism in case of loss of power
all Robotics arms are integrated with force sensors that can detect excessive forces and stop immediately
speed of movement is limited during treatment to below 3000 mm/sec and below 50 mm/sec while moving from one coring location to another
movements during coring are limited to 20 mm and maximum allowed orientation is less than 10 degrees
Imaging system continuously monitors distance between punches and skin
All computer-controlled movements are initiated by the user. These movements can be quickly stopped via the user interface.

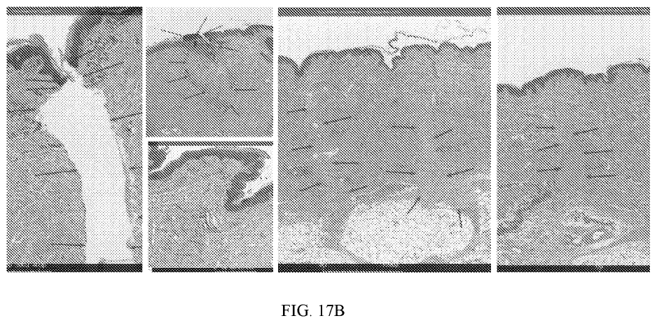

Reference is now made to FIG. 17A illustrating histological analysis-cross tissue sections after 0, 2 and 5 weeks post the fractional coring (tissue removal) treatment.

FIG. 17B illustrates 1, 7, 14 and 28 days post the treatment (the creation of interference). The leftmost part illustrates the interference being formed, necrotic zone, fibroblasts migration (seen on the second slide from left), septae-like scar formation and maturation (between red arrows in two slides at right side).

As can be seen in the figure, immediately after the treatment (at 0 weeks), fractional holes have been created post the excision of the cored tissue.

After 2 and 5 weeks, healing including fibroblasts migration and collagen synthesis as well as maturation occurred and the skin was tightened.

According to another embodiment of the present invention the excised tissue could be according to any embodiment as disclosed above, however, the directional tightening thereof could also be performed by application of at least one energy source being selected from a group consisting of application of temperature to heat and evacuate tissue, application of temperature to heat, application of laser, insertion of threads, application of heat to accelerate collagen synthesis in the tissue, RF, coblation, coagulation, ablation, microwave energy, ultrasound, application of any other type of energy and any combination thereof.

In such an embodiment, for example, an RF electrode could be applied either to the entire treated skin region or to the area between each excised region.

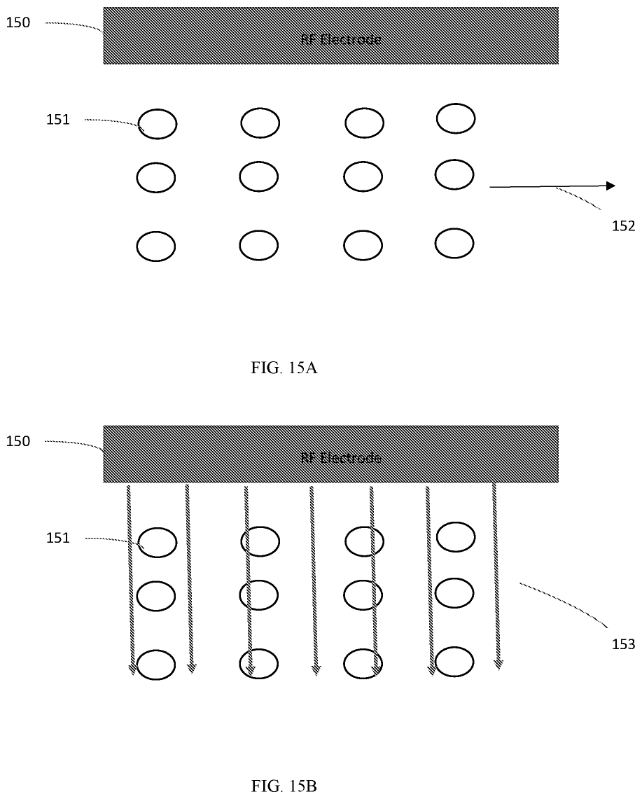

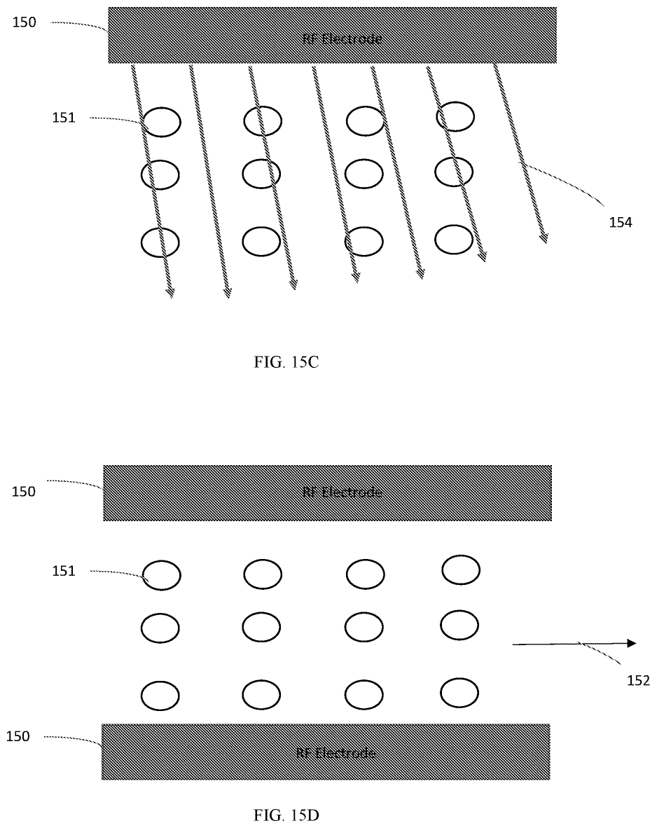

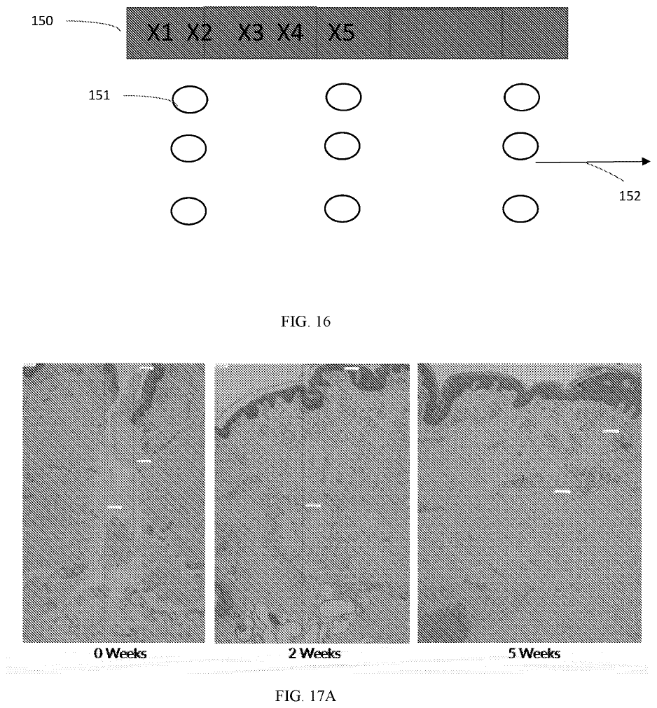

Reference is now made to FIGS. 15-16 schematically illustrating such an embodiment.

In FIG. 15A schematically illustrated the skin region in which plurality of excisions 150 have been produced. In this figure, also integrated is an RF electrode 150 which post the excision are adapted to apply energy to the skin to provide the directional tightening. It is within the scope of the present invention that once the RF energy is applied to the tissue a different magnetic field would be created in between the excised tissue so as to provide skin tightening (see arrow 152).

It should be noted that the energy applied by the RF electrode (or a different energy source) could be e.g., as illustrated in FIG. 15B (see arrow 153) or 15c (see arrow 154).

According to another embodiment, when applicable, 2 RF electrodes are employed (each from a different side of the skin), see FIG. 15D.

Reference is now made to FIG. 16 which schematically illustrates another embodiment of the present invention, in which the energy applied to the skin tissue (in this case RF energy) is divided into several segments (FIG. 16 illustrates 5 segments X1 . . . X5), each section is adapted to apply a different amount of energy to the tissue. Such energy level could be adjusted to optimize the treatment.

It should be emphasized that although FIGS. 15-16 illustrates RF electrode and RF energy, the same applies to application of laser, application of heat to accelerate collagen synthesis in the tissue, RF, pulsed electromagnetic field, insertion of threads, coblation, ablation, coagulation, microwave energy, ultrasound, application of any other type of energy and any combination thereof.

Combined Energy-based Coring with the Interference Means (e.g., the Mechanical-Based Coring)

The following disclosure describe the combination of energy-based coring means with coring means; however, it is emphasized that the combination of energy-based coring means with interference means is within the scope of the present invention.

According to another embodiment of the present invention, the punches/needles are also adapted to apply RF energy to the skin and tissue.

According to such an embodiment, the punches/needles are adapted to penetrate and core the skin (to produce a plurality of excised tissue portions) and either simultaneously or sequentially deliver RF energy to provide heat to the tissue and to fractional ablate/coagulate the tissue. In such an embodiment, the punches/needles are basically an RF electrode as well as a cutting element.

It is within the scope of the present invention, where the application of RF energy to the skin will facilitate the tissue excision as well as apply ablative and coagulative wound healing derived impact to the tissue.

According to one embodiment, each punch/needle is in communication with at least one RF generator.

According to another embodiment, all punches/needles are in communication with at least one RF generator.

According to another embodiment of the present invention, pulsed electromagnetic frequency generator is in communication with at least one of said punches/needles. According to another embodiment, the pulsed electromagnetic frequency generator is adapted to provide a dynamic magnetic field such that electromagnetic pulses are delivered to said region of a patient's skin. According to another embodiment, said electromagnetic pulses vary with time.

According to another embodiment, the dynamic magnetic field is provided by means of at least one coil. According to another embodiment, at least one of the punches/needles is at least partially coiled by at least one coil. According to another embodiment, all the punches/needles are at least partially coiled by one coil.

According to another embodiment of the present invention, all of said punches/needles are adapted to simultaneously provide said electromagnetic pulses to said region of a patient's skin and apply RF energy. According to one embodiment of the present invention said RF energy results in heating said skin.

According to another embodiment of the present invention, application of at least one energy selected from a group consisting of laser, pulsed electromagnetic field, RF, coblation, coagulation, ablation, microwave energy, ultrasound, application of any other type of energy and any combination thereof is either simultaneously or sequentially applied with the coring of said skin.

According to another embodiment of the present invention, a control unit monitors and/or controls said the application of heat (by means of the RF energy) to the tissue within said region of skin.

According to another embodiment of the present invention, the shape of said electromagnetic pulse is selected from the group consisting of square wave, a sine wave, a triangular wave, sawtooth wave, ramp waves, spiked wave or any combination thereof.

According to another embodiment of the present invention, the magnetic field intensity B of each pulse applied by said pulsed electromagnetic frequency generator ranges between about 0 and about 3 Tesla.

According to another embodiment of the present invention, the magnetic field intensity B of each pulse applied by said pulsed electromagnetic frequency generator ranges between about 0 to 40 Gauss.

According to another embodiment of the present invention, the duration of each pulse applied by said pulsed electromagnetic frequency generator ranges between about 3 and about 1000 milliseconds.

According to another embodiment of the present invention, the frequency F applied by the pulses of said pulsed electromagnetic frequency generator ranges between about 1 Hz and about 40 MHz.

According to another embodiment of the present invention, the energy E applied by the pulses of said pulsed electromagnetic frequency generator ranges between about 1 and about 150 watts per pulse or any combination thereof.

According to another embodiment of the present invention, the frequency F applied by the pulses applied by said step of applying pulsed electromagnetic therapy to said region to be higher than about 1 and lower than about 1M Hz.

According to another embodiment of the present invention, the frequency F applied by said electromagnetic field pulses ranges between 1 Hz and 50 Hz.

According to another embodiment of the present invention, the frequency of said RF energy ranges between 200 kHz and 10 MHz.

According to another embodiment of the present invention, the power P applied by said RF energy pulses ranges between 1 W and 100 W of RMS average power.

According to another embodiment of the present invention, at least one temperature sensor is provided.

According to another embodiment of the present invention, the temperature T the tissue reaches is higher than about 30 and lower than about 100 degrees.

According to another embodiment of the present invention, a mechanism for skin cooling is provided to regulate the temperature of the skin (applied by the RF energy).

According to another embodiment of the present invention, the device additionally comprising at least one RF electrode (in addition to the coring element; namely, the punches/needles) adapted to apply RF energy to the skin and tissue.

According to such an embodiment, the punches/needles are adapted to penetrate and core the skin (to produce a plurality of excised tissue portions) while the RF electrode either simultaneously or sequentially deliver RF energy to provide heat to the tissue and to fractional ablate/coagulate the tissue.

It is within the scope of the present invention, where the application of RF energy to the skin will facilitate the tissue excision as well as apply ablative and coagulative therapy to the tissue. According to one embodiment, the RF electrode is in communication with at least one RF generator.

According to another embodiment of the present invention, pulsed electromagnetic frequency generator is in communication with the at least one RF electrode. According to another embodiment, the pulsed electromagnetic frequency generator is adapted to provide a dynamic magnetic field such that electromagnetic pulses are delivered to said region of a patient's skin. According to another embodiment, said electromagnetic pulses vary with time.

According to another embodiment, the dynamic magnetic field is provided by means of at least one coil. According to another embodiment, at least one of the RF electrodes is at least partially coiled by at least one coil. According to another embodiment, all the RF electrodes are at least partially coiled by one coil.

According to another embodiment of the present invention, all of said RF electrodes are adapted to simultaneously provide said electromagnetic pulses to said region of a patient's skin and apply Rf energy. According to one embodiment of the present invention said RF energy results in heating said skin.

According to another embodiment of the present invention, a control unit monitors and/or controls said the application of heat (by means of the RF energy) to the tissue within said region of skin.

According to another embodiment of the present invention, the shape of said electromagnetic pulse is selected from the group consisting of square wave, a sine wave, a triangular wave, sawtooth wave, ramp waves, spiked wave or any combination thereof.

According to another embodiment of the present invention, the magnetic field intensity B of each pulse applied by said pulsed electromagnetic frequency generator ranges between about 0 and about 3 Tesla.

According to another embodiment of the present invention, the magnetic field intensity B of each pulse applied by said pulsed electromagnetic frequency generator ranges between about 0 to 40 Gauss.

According to another embodiment of the present invention, the duration of each pulse applied by said pulsed electromagnetic frequency generator ranges between about 3 and about 1000 milliseconds.

According to another embodiment of the present invention, the frequency F applied by the pulses of said pulsed electromagnetic frequency generator ranges between about 1 Hz and about 40 MHz.

According to another embodiment of the present invention, the energy E applied by the pulses of said pulsed electromagnetic frequency generator ranges between about 1 and about 150 watts per pulse or any combination thereof.

According to another embodiment of the present invention, the frequency F applied by the pulses applied by said step of applying pulsed electromagnetic therapy to said region to be higher than about 1 and lower than about 1M Hz.

According to another embodiment of the present invention, the frequency F applied by said electromagnetic field pulses ranges between 1 Hz and 50 Hz.

According to another embodiment of the present invention, the frequency of said RF energy ranges between 200 kHz and 10 MHz.

According to another embodiment of the present invention, the power P applied by said RF energy pulses ranges between 1 W and 100 W of RMS average power.

According to another embodiment of the present invention, at least one temperature sensor is provided.

According to another embodiment of the present invention, the temperature T the tissue reaches is higher than about 30 and lower than about 100 degrees.

According to another embodiment of the present invention, a mechanism for skin cooling is provided to regulate the temperature of the skin (applied by the RF energy).

### Impedance/Temperature Measurements

According to another embodiment of the present invention, at least one impedance/temperature sensor(s) is embedded in the distal-most end of at least one of the interference means (e.g., punches/needles) to provide indication as to the depth of penetration of each of at least one of the punches. Such information can be utilized to indicate if each punch is within the preferred treatment zone or outside thereof.

### Cutting Element

According to another embodiment of the present invention, the skin interference instrument (e.g., the punches/needles) comprise at least one cutting element (e.g., at least one blade), adapted to grind/mil the cored/excised tissue so as to facilitate extraction thereof.

The at least one cutting element could be integrated in the interference means (e.g., punches/needles) or in communication therewith.

As stated above, according to one object of the present invention, the system comprises at least one vacuum subsystem adapted to apply suction to remove excising portions of said skin tissue. Combining the at least one cutting element in the system will facilitate the extraction of the excised tissue by said vacuum subsystem. Alternatively, the cutting element will facilitate the removal of the cored/excised tissue with the aid of the retention member.

### Injectable Matter

According to another embodiment of the present invention, at least one needle is provided with the punches, to inject treatment substances to the treatment area.

According to another embodiment of the present invention, the punches are needles adapted to inject treatment substances to the treatment area.

According to another embodiment of the present invention, the needles could be with either of a homogeneous/heterogeneous size.

According to another embodiment of the present invention, the substance could be selected from a group consisting of hyaluronic acid, botox, collagen, stem cells or any of the adhesives described above.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

1-135. (canceled)

136. A method for treating cellulite on a patient's skin, comprising:
i. identifying at least one region of said patient's skin with cellulite; and
ii. forming at least one interference of at least one tissue portion in said region of skin, thereby generating at least one septa in said region of said patient's skin so as to treat the cellulite.

137. The method of claim 136, wherein said step of forming at least one interference in said tissue portion comprises at least one step selected from a group consisting of excising at least one tissue portion; coring at least one tissue portion; incising of at least one tissue portion, providing at least one additive to said skin, and any combination thereof.

138. The method of claim 136, wherein said step of forming at least one interference in a portion of said tissue portion results in generating a plurality of septa in said region of skin, each at a different angle, relative to each other and said skin.

139. The method of claim 136, wherein said step of forming at least one interference of at least one tissue portion is performed at an angle A with respect to the surface of said region of skin, wherein said angle A is in the range of about 0 to about 90 degrees.

140. The method of claim 136, wherein said step of forming at least one interference of at least one tissue portion results in the generation of a cruciform structure in said at least a tissue portion.

141. The method of claim 136, further comprising step of confirming that the generated septa is associated with the alleviation of expression of cellulite.

142. The method of claim 136, additionally comprising step of applying contraction or expansion tension to said region of skin tissue before and/or after said step of forming at least one interference of at least one tissue portion, wherein said step of applying tension therebetween said two portions applies force in the range of 0 N/mm²-50 N/mm².

143. The method of claim 136, wherein said step of forming at least one interference of at least one tissue portion in said region of skin is performed by a system comprising at least one robotic arm, said at least one robotic arm comprising at least one skin interference instrument.

144. The method of claim 143, wherein the at least one skin interference instrument comprises a plurality of skin interference instruments and wherein the distance between each pair of neighboring skin interference instruments is configured to vary and be adjustable either before or during treatment.

145. The method of claim 143, wherein said system additionally comprises at least one imaging subsystem adapted to guide said at least one skin interference instrument, wherein said imaging subsystem comprises at least one selected from a group consisting at least one camera, under-skin imaging such as ultrasound-based imaging, OCT and any combination thereof.

146. The method of claim 136, additionally comprising providing at least one treatment substance to the treatment area.

147. A system of treating cellulite on a patient's skin, comprising:
(i) a system including at least one robotic arm for identifying at least one region of said patient's skin with cellulite; and,
(ii) means for forming at least one interference of at least one tissue portion of skin thereby generating at least one septa in said region of said patient's skin;
wherein said at least one septa generated in said region of said patient's skin treats cellulite.

148. The system of claim 147, wherein said at least one robotic arm comprises at least one skin interference instrument.

149. The system of claim 148, wherein said at least one skin interference instrument comprises a plurality of skin interference instruments that are adapted to penetrate said skin either in a simultaneously or sequentially manner.

150. The system of claim 149, wherein said plurality of skin interference instruments are characterized by either a similar or substantially different cross section area.

151. The system of claim 149, wherein said plurality of skin interference instruments are adapted to penetrate said skin to a depth of 1 to 20 mm or wherein at least a portion of said plurality of skin interference instruments are characterized by a diameter of 0.15 mm-2.0 mm.

152. The system of claim 150, wherein said cross section area is selected from a group consisting of circular, rectangular, triangular, hexagonal, oval, staggered rows, parallel rows, a spiral pattern, a square or rectangular pattern, a radial distribution and any combination thereof.

153. The system of claim 150, wherein said system additionally comprises at least one controller adapted to control the positioning and orientation of said at least one robotic arm relatively to said skin area.

154. The system of claim 153, wherein said controller is adapted to control at least one parameter selected from a group consisting of the rotation, translation, angle of said at least one robotic arm relatively to said skin, location of impact, depth of penetration, coverage rate, the diameter of at least one excised tissue multiplied by a quantity of cores, area of said skin to be treated, and any combination thereof, wherein said at least one parameter is (i) adjusted manually by the operator or automatically by said controller or (ii) adjustable in real time.

155. The system of claim 149, wherein said plurality of skin interference instruments move by either (i) rotating or (ii) translating, wherein the rotation is at a rotational speed in a range of 1000-10000 RPM, or the translation is at a translation speed is in a range of 0-3000 mm/sec.

156. The system of claim 149, wherein said plurality of skin interference instruments move simultaneously or individually.

157. The system of claim 149, wherein said angle of said at least one robotic arm to said skin is in the range of about 0 to about 90 degrees.

158. The system of claim 153, wherein said controller is adapted to define at least one no-fly zone; said no-fly zone being an area to which said system provides no treatment.

159. The system of claim 149, wherein said at least one skin interference instrument is in communication with at least one RF generator, adapted to apply RF energy to the skin and tissue, so as to fractionally ablate/coagulate the tissue, wherein said application of RF energy is either simultaneously or sequentially with said at least one skin interference instrument.

160. The system of claim 149, wherein one or more of said plurality of skin interference instruments is (i) at least partially coiled by at least one coil, (ii) additionally comprises at least one needle to provide at least one treatment substance to the treatment area, or (iii) is adapted to at least partially sever at least one septum.
